(19) United States
(12) Patent Application Publication
(10) Pub. No.: US 2024/0180986 A1
(43) Pub. Date: Jun. 6, 2024

(54) COMPOSITIONS AND METHODS FOR TREATING AND PREVENTING GASTROINTESTINAL INFLAMMATION

(71) Applicant: DUPONT NUTRITION BIOSCIENCES APS, Kongens Lyngby (DK)

(72) Inventors: Patrick Boyaval, La Meziere (FR); Sebastien Guery, Niebüll (DE); Mathias Richard, Saint-Cyr-lEcole (FR)

(21) Appl. No.: 18/553,229

(22) PCT Filed: Mar. 30, 2022
(86) PCT No.: PCT/EP2022/058442

§ 371 Date: Sep. 29, 2023

(30) Foreign Application Priority Data
Mar. 30, 2021 (EP) 211658182

(51) Int. Cl. A61K 36/064; A61P 29/00

(52) U.S. Cl. CPC A61K 36/064; A61P 29/00

(57) ABSTRACT

Provided herein are compositions containing fungus, e.g., yeast, useful for treating and/or preventing gastrointestinal inflammation, and/or diseases, disorders, or conditions associated therewith, in a subject in need thereof and methods for using the same.

### FIELD OF THE INVENTION

Provided herein are compositions containing fungus, e.g., yeast, useful for treating and/or preventing gastrointestinal inflammation, and/or diseases, disorders, or conditions associated therewith, in a subject in need thereof and methods for using the same.

### BACKGROUND

Gastrointestinal inflammation is diverse in etiology, pathogenesis, and manifestation. Gastrointestinal inflammation has been associated with a variety of diseases, disorders and conditions, including those of the gastrointestinal tract (e.g., inflammatory bowel disease (IBD)) as well as extra-intestinal diseases, disorders, and conditions. Thus, treatment or prevention of inflammation in the gastrointestinal tract may assist in the treatment and/or prevention of a wide range of diseases, disorders, and conditions not necessarily restricted to the gastrointestinal tract.

Common methods for treating inflammation, such as gastrointestinal inflammation, include anti-inflammatory drugs, immunosuppressants, biologics, and antibiotics. However, such pharmacological treatments may be accompanied by serious side effects, including infection, gastrointestinal lesions, and neoplasia. Furthermore, not all subjects respond to or remain responsive to treatment.

Thus, there remains a need for safe and effective compositions and methods for treating and/or preventing gastrointestinal inflammation, and disease, disorders, and conditions associated with gastrointestinal inflammation. The compositions and methods provided herein address such needs.

### SUMMARY OF THE INVENTION

Provided herein are compositions containing Cyberlindnera jadinii. In some embodiments, the Cyberlindnera jadinii is the strain deposited at the DSMZ [Deutsche Sammlung von Mikroorganismen und Zellkulturen GmbH, Inhoffenstrasse 7B, D-38124 Braunschweig—Germany] under number DSM 33763 or a strain having all of the identifying characteristics of the Cyberlindnera jadinii strain deposited at DSMZ under number DSM 33763. In some embodiments, the strain having all of the identifying characteristics of the Cyberlindnera jadinii strain deposited at DSMZ under number DSM 33763 is a live strain. In some embodiments, the Cyberlindnera jadinii is dried. In some embodiments, the Cyberlindnera jadinii is lyophilized. In some embodiments, the Cyberlindnera jadinii is spray dried. In some embodiments, the Cyberlindnera jadinii is drum dried. In some embodiments, the compositions contain an effective amount of the Cyberlindnera jadinii to treat and/or prevent gastrointestinal inflammation and/or a disease, disorder, or condition associated with gastrointestinal inflammation. In some embodiments, the composition contains an amount of the Cyberlindnera jadinii that is at least about 1×10⁹ CFU/g to at least about 5×10¹⁰ CFU/g of composition. In some embodiments, the composition contains an amount of the Cyberlindnera jadinii that is or is about 2.5×10¹⁰ CFU/g of composition. In some embodiments, the amount is an effective amount.

Also provided herein are compositions containing Kluyveromyces lactis. In some embodiments, the Kluyveromyces lactis is the strain deposited at the DSMZ [Deutsche Sammlung von Mikroorganismen und Zellkulturen GmbH, Inhoffenstrasse 7B, D-38124 Braunschweig—Germany] under number DSM 33764 or a strain having all of the identifying characteristics of the Kluyveromyces lactis strain deposited at DSMZ under number DSM 33764. In some embodiments, the strain having all of the identifying characteristics of the Kluyveromyces lactis strain deposited at DSMZ under number DSM 33764 is a live strain. In some embodiments, the Kluyveromyces lactis is dried. In some embodiments, the Kluyveromyces lactis is lyophilized. In some embodiments, the Kluyveromyces lactis is spray dried. In some embodiments, the Kluyveromyces lactis is drum dried. In some embodiments, an effective amount of the Kluyveromyces lactis to treat and/or prevent gastrointestinal inflammation and/or a disease, disorder, or condition associated with gastrointestinal inflammation. In some embodiments, the composition contains an amount of the Kluyveromyces lactis that is at least about 1×10⁹ CFU/g to at least about 5×10¹⁰ CFU/g of composition. In some embodiments, the composition contains an amount of the Kluyveromyces lactis that is or is about 2.5×10¹⁰ CFU/g of composition. In some embodiments, the amount is an effective amount.

In some embodiments, the composition is a probiotic. In some embodiments, the composition is a pharmaceutical composition and further contains at least one pharmaceutically acceptable carrier and/or excipient. In some embodiments, the composition is formulated for oral administration. In some embodiments, the composition is encapsulated or coated. In some embodiments, the composition is a food product, food ingredient, dietary supplement, or medicament. In some embodiments, the composition is formulated for topical administration.

In some embodiments, the composition treats and/or prevents gastrointestinal inflammation and/or a disease, disorder, or condition associated with gastrointestinal inflammation. In some embodiments, the disease, disorder, or condition is IBD, IBS, cancer, chemotherapy-induced gastrointestinal inflammation, radiation-induced gastrointestinal inflammation, immunotherapy-induced gastrointestinal inflammation, bacterial infection, viral infection, fungal infection, antibiotic use, aging, dysbiosis, obesity, type 2 diabetes, metabolic syndrome, asthma, atherosclerosis, non-alcoholic fatty liver disease, multiple sclerosis, or skin inflammation.

Provided herein are tablets, chewable gums, capsules, granules, powders, sachets, patches, creams, or ointments containing the composition described herein.

Provided herein are kits including the composition provided herein or a tablet, chewable gum, capsule, granule, powder, sachet, patch, cream, or ointment provided herein and written instructions for administration to a subject.

Also provided herein are methods for treating and/or preventing gastrointestinal inflammation and/or a disease, disorder, or condition associated with gastrointestinal inflammation, in a subject in need thereof, including administering an effective amount of the composition described herein or a tablet, chewable gum, capsule, granule, powder, sachet, patch, cream, or ointment of as described herein to the subject. In some embodiments, the disease, disorder, or condition treated and/or prevented is IBD, IBS, cancer, chemotherapy-induced gastrointestinal inflammation, radiation-induced gastrointestinal inflammation, immunotherapy-induced gastrointestinal inflammation, bacterial infection, viral infection, fungal infection, antibiotic use, aging, dysbiosis, obesity, type 2 diabetes, metabolic syndrome, asthma, atherosclerosis, non-alcoholic fatty liver disease, multiple sclerosis, or skin inflammation. In some embodiments, the disease is IBD. In some embodiments, the IBD is ulcerative colitis or Crohn's disease. In some embodiments, the disease, disorder or condition is dysbiosis.

Provided herein are compositions for use in the treatment and/or prevention of gastrointestinal inflammation and/or a disease, disorder, or condition associated with gastrointestinal inflammation in a subject in need thereof, including the composition described herein or the tablet, chewable gum, capsule, granule, powder, sachet, patch, cream, or ointment described herein. In some embodiments, the disease, disorder, or condition is IBD, IBS, cancer, chemotherapy-induced gastrointestinal inflammation, radiation-induced gastrointestinal inflammation, immunotherapy-induced gastrointestinal inflammation, bacterial infection, viral infection, fungal infection, antibiotic use, aging, dysbiosis, obesity, type 2 diabetes, metabolic syndrome, asthma, atherosclerosis, non-alcoholic fatty liver disease, multiple sclerosis, or skin inflammation. In some embodiments, the disease, disorder, or condition is IBD. In some embodiments, the IBD is ulcerative colitis or Crohn's disease. In some embodiments, the disease, disorder, or condition is dysbiosis.

Each of the aspects and embodiments described herein are capable of being used together, unless excluded either explicitly or clearly from the context of the embodiment or aspect.

### BRIEF DESCRIPTION OF THE FIGURES

FIGS. 1A-1B show yeast survival in the mouse gut environment at initiation of Dextran Sodium Sulfate (DSS) treatment to induce colitis (day 0) (FIG. 1A) and at day 12 (FIG. 1B). Yeast and DSS treatment regimens and quantification of colony forming units (CFU) are described in Example 1. One-way ANOVA, *p<0.05, **p<0.01.

FIGS. 2A-2B show mouse body weight, expressed as a percentage of the initial body weight at the onset of DSS-treatment, over time in animals treated with yeast or phosphate buffered saline (PBS) used for yeast cell suspension (vehicle). FIG. 2A shows data for mice treated with C. jadinii, K. lactis, or vehicle (PBS). FIG. 2B shows data for mice treated with K. unispora, P. membranifaciens, S. cerevisiae, D. hansenii, or vehicle (PBS). Yeast and DSS treatment regimens are described in Example 1.

FIGS. 3A-3B show disease activity index (DAI) scores over time in DSS-induced colitis mice treated with yeast as shown or vehicle. FIG. 3A shows data for mice treated with C. jadinii, K. lactis, or vehicle (PBS). FIG. 3B shows data for mice treated with K. unispora, P. membranifaciens, S. cerevisiae, D. hansenii, or vehicle (PBS). DAI was calculated as a total score: body weight decrease+stool consistency+rectal bleeding divided by 3. Vehicle was the phosphate buffered saline used for yeast cell suspensions. Yeast and DSS treatment regimens are described in Example 1.

FIG. 4 shows body weight loss (expressed as a percentage of the initial body weight at the onset of DSS-treatment to induce colitis) in DSS-induced colitis mice treated with C. jadinii, K. lactis, cyclosporin, or vehicle (PBS). Statistical significance was determined by two-way ANOVA and p-values are denoted as follows: *p<0.05; **p<0.005; ****p<0.0001 Yeast, cyclosporin, and DSS treatment regimens are described in Example 1.

### DETAILED DESCRIPTION OF THE INVENTION

Gastrointestinal inflammation refers to the complex immunological response mounted to protect against injury to the gastrointestinal tract. Gastrointestinal injury may arise from pathogens, cellular damage, and/or alterations in the gastrointestinal microbiome (dysbiosis) that initiate an immune response. The role of dysbiosis in the etiology and pathogenesis of gastrointestinal inflammation has received particular attention in view of evidence suggesting correlations between gastrointestinal microbiota and a variety of diseases and disorders, such as infection, autoimmune disease, cancer, and metabolic and neurodegenerative disorders, many of which are associated with inflammation.

The gastrointestinal microbiome includes bacteria, viruses, and fungi, that, when present in a favorable balance, may promote food digestion, xenobiotic metabolism, and regulate innate and adaptive immunological processes. There is an emerging appreciation of the wide-spread and complex roles that gastrointestinal microbiota, and functions thereof, play at distal sites within a host, such as in the lung, brain, liver, and skin. Indeed, gastrointestinal dysbiosis has been implicated in local and systemic inflammation associated with a diverse set of diseases, disorders, and conditions.

Despite fungus constituting only a fraction of the microbiome, fungal dysbiosis has been implicated in a variety of diseases, including inflammatory gastrointestinal disorders. Mechanistic studies have suggested that enteric fungi, such as pathogenic or opportunistic yeasts and molds, may contribute to initiation of gut inflammation and the pathogenesis of inflammatory bowel disease (IBD).

The ability of gastrointestinal microbiota, such as yeast, to influence immunological responses suggests that manipulation of gastrointestinal microbiota may be useful for treating and/or preventing gastrointestinal inflammation, which may result in the treatment or prevention of diseases, disorders, and conditions associated therewith.

As described herein, it was surprisingly found that yeasts could treat or prevent gastrointestinal inflammation and diseases, disorders, and conditions associated therewith. For example, it was surprisingly found that administration of yeasts, such as Cyberlindnera jadinii (anamorph of Candida utlilis) and Kluyveromyces lactis, reduced symptoms of the inflammatory bowel disease ulcerative colitis (UC) in an animal model (see, Section V).

It was also surprising found that administered yeasts could persist in the gastrointestinal tract. For example, when Cyberlindnera jadinii was administered for treatment of UC in an animal model, the yeast survived in the intestinal environment for at least 12 days (see, Section V). This suggests that yeast treatment could have long-lasting protective effects via intestinal colonization.

The potential to administer yeasts to treat or prevent gastrointestinal inflammation, and diseases, disorders, and conditions associated therewith, is significant given that the side effects of current pharmacological treatments for gastrointestinal inflammation can be severe, e.g., including lesions, infection, and neoplasia. In some embodiments, the yeasts contemplated for use herein have a history of safe use in food products.

Yeasts also have an advantage in that they are more robust than bacteria to temperature, pH, osmotic pressure, oxygen (e.g., dioxygen), and alcohols. Therefore, yeasts are more stable compared to bacteria, which may allow for more effective oral or topical administration. For example, yeasts are resistant to low pH, e.g., yeast can grow at pH levels such as pH 3.0, which allows them to survive the acidic conditions in the stomach to reach the intestines. Such properties are advantageous for delivering compositions described herein to the gastrointestinal tract, including the intestines.

Thus, the compositions and methods provided herein harness the surprising findings described herein to offer new strategies for treating and/or preventing gastrointestinal inflammation, and diseases, disorders, and conditions associated therewith.

This disclosure is not limited by the exemplary methods and materials disclosed herein, and any methods and materials similar or equivalent to those described herein can be used in the practice or testing of embodiments of this disclosure.

The headings provided herein are not limitations of the various aspects or embodiments of this disclosure which can be had by reference to the specification as a whole. The section headings used herein are for organizational purposes only and are not to be construed as limiting the subject matter described. Any terms defined are more fully defined by reference to the specification as a whole.

The publications discussed herein are provided solely for their disclosure prior to the filing date of the present application. Nothing herein is to be construed as an admission that such publications constitute prior art to the claims appended hereto.

All publications, including patent documents, scientific articles and databases, referred to in this application are incorporated by reference in their entirety for all purposes to the same extent as if each individual publication were individually incorporated by reference.

### 1. DEFINITIONS

As used herein, “microorganism” or “microbe” refers to a bacterium, a fungus, a virus, a protozoan, and other microbes or microscopic organisms.

As used here in the term “probiotic” refers to a composition for consumption by humans (e.g., as an or as a component of food) that contains viable (i.e. live) microorganisms, i.e., microorganisms that are capable of living and reproducing that, when administered in adequate amounts, confer a health benefit on a subject (see Hill et al. 2014 Nature Revs Gastro & Hep 11, 506-514, incorporated by reference herein in its entirety). A probiotic may contain one or more (such as any of 1, 2, 3, or 4) of any of the yeasts and strains thereof described herein. Probiotics are distinguished from yeast compositions that have been killed, for example, by pasteurization or heat treatment. Administration of non-viable yeast compositions for the treatment of gastrointestinal inflammation and diseases, disorders, and conditions associated therewith is also contemplated in certain embodiments of the methods disclosed herein.

A yeast “strain” as used herein refers to a yeast which remains genetically and functionally unchanged when grown or multiplied. The multiplicity of identical yeast is included. In some cases, “genetically unchanged” as used herein may include the presence of single nucleotide polymorphisms (SNPs) and/or mutations in the yeast genome, for example SNPs and mutations that occur naturally during growth of the organism (e.g., multiplication), that do not change the functional properties of the yeast.

By “at least one strain,” is meant a single strain but also mixtures of strains comprising at least two strains of microorganisms, e.g., yeasts. By “a mixture of at least two strains,” is meant a mixture of two, three, four, five, six or even more strains. In some embodiments of a mixture of strains, the proportions can vary from 1% to 99%. When a mixture comprises more than two strains, the strains can be present in substantially equal proportions in the mixture or in different proportions.

For purposes of this disclosure, a “biologically pure strain” means a strain containing no other yeast strains in quantities sufficient to interfere with replication of the strain or to be detectable when assessed using techniques recognized in the field.

“Isolated” when used in connection with the organisms and cultures described herein includes not only a biologically pure strain, but also any culture of organisms which is grown or maintained other than as it is found in nature. In some embodiments, the strains are mutants, variants, or derivatives of strains of Cyberlindnera jadinii and/or Kluyveromyces lactis described herein that also provide benefits comparable to that provided by deposited strains of Cyberlindnera jadinii and/or Kluyveromyces lactis as described herein. In some embodiments, the strains are strains having all of the identifying characteristics of the deposited strains of Cyberlindnera jadinii or Kluyveromyces lactis described herein. Further, each individual strain (e.g., Cyberlindnera jadinii strain and Kluyveromyces lactis strain described herein) or any combination of these strains can also provide one or more of the benefits described herein. It will also be clear that addition of microbial strains, carriers, additives (e.g., cryoprotectants, extracts, prebiotics, postbiotics, parabiotics), enzymes, other yeasts, or the like may also provide one or more benefits or improvement of gastrointestinal inflammation and diseases, disorders, and conditions associated therewith, in a subject and will not constitute a substantially different yeast strain.

The term “sequence identity” or “sequence similarity” as used herein, means that two polynucleotide sequences, a candidate sequence and a reference sequence, are identical (i.e. 100% sequence identity) or similar (i.e. on a nucleotide-by-nucleotide basis) over the length of the candidate sequence. In comparing a candidate sequence to a reference sequence, the candidate sequence may comprise additions or deletions (i.e. gaps) as compared to the reference sequence (which does not comprise additions or deletions) for optimal alignment of the two sequences. Optimal alignment of sequences for determining sequence identity may be conducted using the any number of publicly available local alignment algorithms known in the art such as ALIGN or Megalign (DNASTAR), or by inspection.

The term “percent (%) sequence identity” or “percent (%) sequence similarity,” as used herein with respect to a reference sequence is defined as the percentage of nucleotide residues in a candidate sequence that are identical to the residues in the reference polynucleotide sequence after optimal alignment of the sequences and introducing gaps, if necessary, to achieve the maximum percent sequence identity.

As used herein, the term “subject” or “patient” is meant a human. In some embodiments, a subject is suffering from a disease, disorder or condition such as, without limitation, inflammatory bowel disease (IBD), such as ulcerative colitis (UC), Crohn's disease (CD), indeterminant colitis (IC); irritable bowel syndrome (IBS); cancers of different parts of the digestive tracts; side effects of cancer treatments, such as chemotherapy, radiation therapy (also referred to as radiotherapy), and immunotherapy, such as immune checkpoint inhibitor therapy; infection, such as bacterial, fungal or viral infection; symptoms of antibiotic use, e.g., misuse; inflammation associated with aging; dysbiosis; obesity; type 2 diabetes; metabolic syndrome; asthma; atherosclerosis; non-alcoholic fatty liver disease; multiple sclerosis; and skin inflammation, for example skin inflammation associated with gastrointestinal inflammation. In some embodiments, a subject is susceptible to a disease, disorder, or condition described herein. In some embodiments, a subject displays one or more symptoms or characteristics of a disease, disorder, or condition described herein. In some embodiments, a subject does not display any symptom or characteristic of a disease, disorder, or condition described herein. In some embodiments, a subject is someone with one or more features characteristic of susceptibility to or risk of a disease, disorder, or condition described herein. In some embodiments, a subject is a patient. In some embodiments, a subject is an individual to whom diagnosis and/or therapy is and/or has been administered. In some embodiments, the disease, disorder or condition is any disease, disorder, or condition described in Section II-A below.

As used herein, “prevent,” “preventing,” “prevention” and grammatical variations thereof refer to a method of partially or completely delaying or precluding the onset or recurrence of a disease, disorder, or condition, e.g., as described herein, and/or one or more of its attendant symptoms or barring a subject from acquiring or reacquiring (e.g., relapsing) a disease, disorder, or condition or reducing a subject's risk of acquiring or reacquiring (e.g., relapsing) a disease, disorder or condition or one or more of its attendant symptoms. a disease, disorder or condition. In some embodiments, the disease, disorder, or condition and/or one or more of its attendant symptoms or conditions is described in Section II-A below.

As used herein, the terms “reduce”, “reduced”, and “reducing” and variants thereof in relation to a particular trait, characteristic, feature, biological process, or phenomena refers to a decrease in the particular trait, characteristic, feature, biological process, or phenomena. The trait, characteristic, feature, biological process, or phenomena can be decreased by 5%, 10%, 15%, 20%, 25%, 30%, 35%, 40%, 45%, 50%, 55%, 60%, 65%, 70%, 75%, 80%, 85%, 90%, 95%, 100% or greater than 100%. In some embodiments, the particular trait, characteristic, feature, biological process, or phenomena is a symptom or complication of a disease, disorder, or condition described herein, for example in Section II-A. In some cases, reducing includes treating a disease, disorder, condition, symptom, or complication.

As used herein, the term “infection” refers to the invasion and multiplication of pathogenic microorganisms in the body.

As used herein “administer”, “administered”, or “administering” and the like are meant the action of introducing one or more compositions comprising one or more yeast species or strain as described herein, to a subject, such as by feeding or consuming orally. In some embodiments, the administration is topical. When used herein, the term topical includes references to formulations that are adapted for application to body surfaces (e.g. the skin or mucous membranes). The composition containing one or more yeasts, e.g., yeast strains, as described herein can also be administered in one or more doses.

The terms effective amount or therapeutically effective amount may refer to a quantity of a composition containing yeast as described herein to improve one or more metrics in subject having gastrointestinal inflammation and diseases, disorders, and conditions associated therewith. In some embodiments, the effective or therapeutic amount is an amount of yeast as described herein to improve one or more metrics in a subject having gastrointestinal inflammation and diseases, disorders, and conditions associated therewith. In some embodiments, the effective or therapeutic amount is an amount of yeast described herein. In some embodiments, the effective or therapeutic amount is an amount of a composition containing yeast described herein.

In some embodiments, an effective or therapeutic amount is amount of yeast or a composition containing yeast that when administered at least once improves one or more metrics in subject having gastrointestinal inflammation and diseases, disorders, and conditions associated therewith. In some embodiments, an effective or therapeutic amount is amount of yeast or a composition containing yeast that when administered more than once, e.g., two or more times, improve one or more metrics in subject having gastrointestinal inflammation and diseases, disorders, and conditions associated therewith. Improvement in one or more metrics of a subject (such as, without limitation, any of treating and/or preventing of gastrointestinal inflammation, and diseases, disorders, and conditions associated therewith) can be measured as described herein or by other methods known in the art.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In some embodiments, the term “consisting essentially of,” as used herein may refer to a composition wherein the component(s) after the term is in the presence of other known component(s) in a total amount that is less than 30% by weight of the total composition and do not contribute to or interferes with the actions or activities of the component(s).

The terms “comprising”, “comprises,” “comprise” and “comprised of” as used herein are synonymous with “include,” “including”, “includes” or “containing”, “contains”, “contain” and are inclusive or open-ended and do not exclude additional, non-recited members, elements or method steps. The terms “comprising”, “comprises,” “comprise,” and “comprised of” also include the term “consisting of”.

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein.

Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

### I. COMPOSITIONS

Provided herein are yeast and compositions containing yeast useful for the treatment and/or prevention of gastrointestinal inflammation. In some embodiments, the yeast and compositions containing yeast useful for the treatment and/or prevention of the gastrointestinal inflammation are useful for the treatment and/or prevention of diseases, disorders, and conditions associated with gastrointestinal inflammation. A disease, disorder, or conditions may be considered associated with gastrointestinal inflammation if the gastrointestinal inflammation is detected, present, correlated, or has an otherwise observable relationship, e.g., a quantitatively or qualitatively observable relationship, with the disease, disorder, or condition. In some embodiments, the gastrointestinal inflammation may be a symptom of the disease, disorder, or condition. In some embodiments, the gastrointestinal inflammation may contribute to or facilitate the pathogenesis of the disease, disorder, or condition. In some embodiments, the gastrointestinal inflammation may contribute to or facilitate the development of the disease, disorder, or condition. In some embodiments, the diseases, disorders, and conditions associated with gastrointestinal inflammation include IBD, such as UC, CD, IC; IBS; cancers of different parts of the digestive tracts; side effects of cancer treatments, such as chemotherapy, radiation therapy, and immunotherapy, such as immune checkpoint inhibitor therapy; infection, such as bacterial, fungal or viral infection; symptoms of antibiotic use, including misuse; inflammation associated with aging; dysbiosis; obesity; type 2 diabetes; metabolic syndrome; asthma; atherosclerosis; non-alcoholic fatty liver disease; multiple sclerosis; and skin inflammation, for example skin inflammation associated with gastrointestinal inflammation. In some embodiments, the diseases, disorders, and conditions associated with gastrointestinal inflammation are or include those described in Section II-A below. In some embodiments, the compositions provided herein treat and/or prevent gastrointestinal inflammation. In some embodiments, the compositions provided herein treat and/or prevent diseases, disorders, or conditions associated with gastrointestinal inflammation. For example, by treating and/or preventing the gastrointestinal inflammation, the associated disease, disorder, or condition is also treated and/or prevented. In some embodiments, the compositions provided herein treat and/or prevent IBD, such as UC.

In some embodiments, the compositions disclosed herein can be used as supplements, food additives, and/or therapeutics for administration to subjects when under periods of physiologic stress (e.g., gastrointestinal inflammation, and diseases, disorders, and conditions associated therewith) or as a part of a daily nutritional regimen to prevent disease (e.g., gastrointestinal inflammation, and diseases, disorders, and conditions associated therewith) and facilitate a healthy digestive tract. In some embodiments, the compositions described herein may be used to treat or prevent gastrointestinal inflammation. In some embodiments, the compositions described herein may be used to treat or prevent disease, disorders, or conditions associated with gastrointestinal inflammation, for example as described herein (see, e.g., Section II-A). In some embodiments, the compositions described herein may be used to treat or prevent symptoms of disease, disorders, or conditions associated with gastrointestinal inflammation, for example as described herein (see, e.g., Section II-A).

Probiotics is another term that can be used to describe the compositions containing yeast, e.g., viable yeast, provided herein. The term “viable” refers to a microorganism, e.g., yeast, which is metabolically active and/or able to multiply. In some embodiments, the compositions disclosed herein include both viable probiotic products and/or compositions that include non-viable yeast or bacteria (such as heat-treated or pasteurized compositions). In some embodiments, the compositions disclosed herein include compositions that contain components of yeast cells. For example, in some cases, the compositions provided herein contain components of the yeast cell wall, cell membrane, and/or intracellular cellular components of yeasts provided herein. In some embodiments, the compositions provided herein are probiotics.

In some embodiments, the compositions provided herein contain yeast from one or more genera. For example, 2, 3, 4, 5, 6, or more different genera. In some embodiments, the compositions provided herein contain species of yeast from one or more genera. For example, one or more species from 2, 3, 4, 5, 6, or more genera. In some embodiments, the compositions provided herein contain strains of a species of yeast from one or more genera. For example, one or more strains of a species from 2, 3, 4, 5, 6, or more genera. In some embodiments, the compositions provided herein contain yeast from a single genus. In some embodiments, the compositions provided herein contain species of yeast from a single genus. For example, 2, 3, 4, 5, 6, or more species of yeast from a single genus. In some embodiments, the compositions provided herein contain one or more strains of a species of yeast from a single genus. For example, 2, 3, 4, 5, 6, or more different strains of a yeast species from a single genus. In some embodiments, the compositions provided herein contain a single strain of a yeast species, e.g., a biologically pure strain. In some embodiments, the yeast genera, species, and strains are any of those described herein, for example in Section I-A below.

### A. Yeasts

The yeasts and compositions containing yeast provided herein may include yeasts that have a history of safe use in food products, or in production thereof, e.g., dairy products such as cheese.

In some embodiments, the yeast is of the genera Cyberlindnera or Kluyveromyces. In some embodiments, the yeast is of the genus Cyberlindnera. In some embodiments, the yeast is of the genus Kluyveromyces. In some embodiments, the yeast is of the species Cyberlindnera jadinii (C. jadinii) or Kluyveromyces lactis (K. lactis). In some embodiments, the yeast is C. jadinii. In some embodiments, the yeast is K. lactis. In some embodiments, the yeast is a strain of C. jadinii or K. lactis. In some embodiments, the yeast is a strain of C. jadinii. In some embodiments, the yeast is a strain of K. lactis.

In some embodiments, the compositions provided herein include yeasts of the genera Cyberlindnera or Kluyveromyces. In some embodiments, the compositions provided herein include yeasts of the genus Cyberlindnera. In some embodiments, the compositions provided herein include yeasts of the genus Kluyveromyces. In some embodiments, the compositions provided herein may include yeasts of the genera Cyberlindnera and Kluyveromyces. In some embodiments, the compositions provided herein may include yeasts of the species Cyberlindnera jadinii (C. jadinii) or Kluyveromyces lactis (K. lactis). In some embodiments, the composition contains C. jadinii. In some embodiments, the composition contains K. lactis. In some embodiments, the compositions provided herein include yeasts of the species C. jadinii and K. lactis. In some embodiments, the composition contains a strain of C. jadinii or K. lactis. In some embodiments, the composition contains a strain of C. jadinii. In some embodiments, the composition contains a strain of K. lactis. In some embodiments, the composition contains a strain of C. jadinii and a strain of K. lactis.

In some embodiments, the strain of C. jadinii is the strain deposited at DSMZ [Deutsche Sammlung von Mikroorganismen und Zellkulturen GmbH, Inhoffenstrasse 7B, D-38124 Braunschweig—Germany] under number DSM 33763. In some embodiments, the strain of C. jadinii is a strain having all of the identifying characteristics of the C. jadinii strain deposited at DSMZ under number DSM 33763. In some embodiments, the strain of C. jadinii having all of the identifying characteristics of the C. jadinii strain deposited at DSMZ under number DSM 33763 is a live strain. In some embodiments, the strain of C. jadinii is a biologically pure strain of the strain DSM 33763. In some embodiments, the C. jadinii strain is a biologically pure strain of C. jadinii that has all of the identifying characteristics of the C. jadinii strain DSM 33763. In some embodiments, the strain of C. jadinii having all of the identifying characteristics of the C. jadinii strain deposited at DSMZ under number DSM 33763 is a live strain.

In some embodiments, the strain of K. lactis is the strain deposited at DSMZ [Deutsche Sammlung von Mikroorganismen und Zellkulturen GmbH, Inhoffenstrasse 7B, D-38124 Braunschweig—Germany] under number DSM 33764. In some embodiments, the strain of K. lactis is a strain having all of the identifying characteristics of the K. lactis strain deposited at DSM under number DSM 33764. In some embodiments, the strain of K. lactis is a biologically pure strain of the strain DSM 33764. In some embodiments, the K. lactis strain is a biologically pure strain of K. lactis that has all of the identifying characteristics of the K. lactis strain DSM 33764. In some embodiments, the strain of K. lactis having all of the identifying characteristics of the K. lactis strain deposited at DSMZ under number DSM 33764 is a live strain.

An identifying characteristic, as used herein, may be a genome sequence percentage identity and/or functional behavior. Functional behavior may be defined by metabolic activity; functional pathways, e.g., signaling pathways; upregulated and downregulated genes, for example as a result of epigenetic changes; protein expression, and protein secretion. In some embodiments, the genome sequence percentage identity is at least 70, 80, 90, 95, 96, 97, 98, 99% or 100%.

### B. Formulations

The compositions disclosed herein, e.g., probiotics, generally contain at least one yeast able to treat or prevent gastrointestinal inflammation and diseases, disorders, and conditions associated therewith as described herein, such as at least one yeast described in Section I-A. In some embodiments, the composition is formulated in freeze-dried (lyophilized) form. For example, the compositions containing yeast can comprise granules or gelatin capsules, for example hard gelatin capsules, including a yeast, e.g., yeast strain, disclosed herein.

In some embodiments, the yeast of the composition described herein are dried. In some embodiments, the compositions disclosed herein contain lyophilized yeast. Lyophilization of yeast is a well-established procedure in the art. In some embodiments, the compositions disclosed herein contain spray dried yeast. Spray drying of yeast is a well-established procedure in the art. In some embodiments, the compositions disclosed herein contain drum dried yeast. Drum drying, in some cases referred to as roll drying, of yeast is a well-established procedure in the art. Alternatively, the compositions may contain a live, active yeast culture.

In some embodiments, the compositions provided herein contain yeast in frozen, dried, freeze-dried, liquid or solid format, in the form of pellets or frozen pellets, or in a powder or dried powder. In some embodiments, the compositions provided herein contain yeast described herein in a frozen format or in the form of pellets or frozen pellets. In some embodiments, the compositions provided herein contain yeast described herein in a dried or freeze-dried format. In some embodiments, the compositions provided herein contain yeast described herein in a powder or dried powder.

In some embodiments, any of the compositions disclosed herein, e.g., probiotics, are encapsulated to enable delivery of the yeast, such as a yeast disclosed herein, to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule.

The compositions disclosed herein can be administered orally and may be in the form of a tablet, capsule, powder, chewable gum, or granule. In some embodiments, the tablet is an effervescent tablet, a chewable tablet, or a lyophilized tablet. In some embodiments, the tablet, capsule, powder, or granule is formulated for prolonged release, e.g., prolonged-release tablet, prolonged-release capsule, or prolonged-release granule. For example, prolonged-release formulations may release the dose over a period of time, such as 2, 4, 6, 8, 10, 12, 14, 16, 18, 20, 22, or 24 hours. Other ingredients (such as vitamin C or minerals, for example), may be included as oxygen scavengers and prebiotic substrates to improve the delivery and/or partial or total colonization and survival in vivo. Alternatively, the compositions (such as probiotic compositions) disclosed herein may be administered orally as a food or nutritional product or as a pharmaceutical product.

In some embodiments, the compositions disclosed herein are formulated as a probiotic. Alternatively, in some embodiments, the compositions disclosed herein are formulated as a non-viable composition, such as a pasteurized or heat-treated yeast composition.

In some embodiments, the composition described herein is administered in the form of a dragee, sachet, or suspension.

The composition, e.g., probiotic, disclosed herein may include a therapeutically effective amount of a yeast disclosed herein (see, e.g., Section I-A). A therapeutically effective amount of a yeast, e.g., yeast strain, is sufficient to exert a beneficial effect upon a subject, e.g., a subject having a disease, disorder, or condition described herein. For example, see Sections II-A and II-B below. In some embodiments, the composition contains an effective amount of Cyberlindnera jadinii and/or Kluyveromyces lactis to treat and/or prevent gastrointestinal inflammation. In some embodiments, the composition contains an effective amount of Cyberlindnera jadinii and/or Kluyveromyces lactis to treat and/or prevent a disease, disorder, or conditions associated with gastrointestinal inflammation as described herein. A therapeutically effective amount of yeast, e.g., yeast strain, may be sufficient to result in delivery to and/or partial or total colonization of the subject's intestine. In some embodiments, therapeutically effective amount of yeast, e.g., yeast strain, is sufficient to survive for a specific duration in a subject's intestine. In some embodiments, the duration is, is at least, is about 6 hours, 12 hours, 24 hours, 2 days, 3 days, 4 days, 5 days, 6 days, 7 days, 10 days, 11 days, 12 days, 13 days, 2 weeks, 3 weeks, one month or longer. In some embodiments, the duration is, is about, or is at least 12 days.

In some embodiments, the composition containing yeast described herein may be administered topically. For example, in some cases, treatment of skin inflammation, e.g., arising from gastrointestinal inflammation and/or related disease, disorders, or conditions, may be accomplished by topical administration. Alternative, in some embodiments, the skin inflammation may be treated or prevented by oral administration of compositions containing yeast described herein.

For topical administration, the compositions containing yeast described herein may be in any form suitable for application to the body surface, such as a cream, lotion, sprays, solution, gel, ointment, paste, patch, plaster, paint, bioadhesive, suspensions or the like, and/or may be prepared so as to contain liposomes, micelles, and/or microspheres. Such a formulation may be used in combination with an occlusive overlayer so that moisture evaporating from the body surface is maintained within the formulation upon application to the body surface and thereafter.

Topical formulations include those in which the active ingredient(s), e.g., yeast, is (are) dissolved or dispersed in a dermatological vehicle known in the art (e.g. aqueous or non-aqueous gels, ointments, water-in-oil or oil-in-water emulsions). Constituents of such vehicles may comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin, natural or synthetic triglycerides such as Miglyol™, or silicone oils such as dimethicone). In some embodiments, the topical compositions include pH buffering agent(s) that when dissolved in a water component of the composition provide a pH in the range of 5 to 7 (e.g., about pH 5.5).

Methods of producing topical compositions, e,g., topical pharmaceutical compositions, such as creams, ointments, lotions, sprays and sterile aqueous solutions or suspensions are well known in the art. Suitable methods of preparing topical pharmaceutical compositions are described, for example in WO 95/10999, U.S. Pat. No. 6,974,585, WO 2006/048747 as well as in documents cited in any of these references.

In some embodiments, the composition contains one or more yeasts as described herein at an amount, independently, of at least or at least about 10⁵, 10⁶, 10⁷, 10⁸, 10⁹, 10¹⁰, or 10¹¹ colony forming units (CFU). In some embodiments, the composition contains one or more yeasts as described herein at an amount, independently, of at least or at least about 10⁹, 10¹⁰, or 10¹¹ CFU.

In some embodiments, the composition contains one or more yeasts as described herein at an amount, independently, of at least or at least about 10¹⁰ CFU. In some In some embodiments, the composition contains one or more yeasts as described herein at an amount, independently, of about 1×10⁵ to about 1×10¹¹ CFU; for example, from about 1×10⁶ to about 1×10¹¹ CFU, from about 1×10⁷ to about 1×10¹¹ CFU, from about 1×10⁸ to about 1×10¹¹ CFU, or from about 1×10⁹ to about 1×10¹¹ CFU, or from about 1×10¹⁰ to about 1×10¹¹ CFU. In some embodiments, the compositions contain one or more yeasts as described herein at an amount, independently, of from or from about 1×10⁸ to about 1×10¹¹ CFU, 1×10⁹ to about 1×10¹¹ CFU, 1×10⁹ to about 1×10¹⁰ CFU. In some embodiments, the compositions contain one or more yeasts as described herein at an amount, independently, from or from about 0.5×10¹⁰ to about 5×10¹⁰ CFU, from or from about 1×10¹⁰ to about 5×10¹⁰ CFU, from or from about 1.5×10¹⁰ to about 5×10¹⁰ CFU, from or from about 2×10¹⁰ to about 5×10¹⁰ CFU, from or from about 2.5×10¹⁰ to about 5×10¹⁰ CFU, from or from about 3×10¹⁰ to about 5×10¹⁰ CFU, from or from about 3.5×10¹⁰ to about 5×10¹⁰ CFU, from or from about 4×10¹⁰ to about 5×10¹⁰ CFU, or from or from about 4.5×10¹⁰ to about 5×10¹⁰ CFU. In some embodiments, the compositions contain one or more yeasts as described herein at an amount, independently, of or of about 1×10¹⁰, 1.5×10¹⁰, 2×10¹⁰, or 2.5×10¹⁰ CFU. In some embodiments, the compositions contain one or more yeasts as described herein at an amount, independently, of or of about 2.5×10¹⁰ CFU. In some embodiments, the CFU may be a CFU per weight, e.g., gram, of composition. Thus, in some of any of the embodiments, the CFU is a CFU per weight of the composition. In some embodiments, the CFU described herein is a CFU per gram of the composition (CFU/g). In some embodiments, the CFU described herein is a CFU per kilogram of the composition (CFU/kg). In some embodiments, the compositions contain one or more yeasts as described herein at an amount, independently, of or of about 2.5×10¹³ CFU/kg.

In some embodiments, the weight of the composition administered to a subject, e.g., human, is between 50 mg and 3000 mg, between 100 mg and 2500 mg, between 150 mg and 2000 mg, between 200 mg and 1500 mg, between 250 mg and 1000 mg, between 300 mg and 950 mg, between 400 mg and 900 mg, between 450 mg and 850 mg, between 500 mg and 800 mg, between 550 mg and 750 mg, or between 600 mg and 700 mg. In some embodiments, the yeast in any of the compositions disclosed herein is administered at a dose of between 200 mg and 3000 mg, between 200 mg and 2500 mg, between 200 mg and 2000 mg, between 200 mg and 1500 mg or between 200 mg and 1000 mg. In some embodiments, the yeast in any of the compositions disclosed herein is administered at a dose of or of about 100 mg, 200 mg, 300 mg, 400 mg, 500 mg, 600 mg, 700 mg, 800 mg, 900 mg, 1000 mg, 1500 mg, 2000 mg, 2500 mg, or 3000 mg. In some embodiments, the yeast in any of the compositions disclosed herein is administered at a dose of or of about 500 mg, 1000 mg, 1500 mg, 2000 mg, 2500 mg, or 3000 mg. In some embodiments, the yeast in any of the compositions disclosed herein is administered at a dose of or of about 100 mg, 200 mg, 300 mg, 400 mg, or 500 mg.

In some embodiments, the composition contains a therapeutically effective amount of Cyberlindnera jadinii and/or Kluyveromyces lactis, e.g., as described herein, to treat or prevent gastrointestinal inflammation and diseases, disorders, and conditions associated therewith as described herein (see, e.g., Section II-A). For example, the composition may contain a therapeutically effective amount of a strain of Cyberlindnera jadinii and/or Kluyveromyces lactis as described herein, effective to treat or prevent an IBD, for example as described in Section II below. In some embodiments, the therapeutically effective amount is at least about 1×10¹⁰ CFU/g to at least about 5×10¹⁰ CFU/g of composition. In some embodiments, the therapeutically effective amount is or is about 2.5×10¹⁰ CFU/g composition.

In some embodiments, the amount of yeast, e.g., as described herein, of the composition is a suitable daily dose for a subject, e.g., human subject. In some embodiments, the subject is a human. In some embodiments, the subject is an adult human. In some embodiments, the subject is a human child. The compositions provided herein may be repeatedly administered to a subject.

Typically, a probiotic is optionally combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

In some embodiments, a probiotic composition described herein is formulated to include a prebiotic compound in an amount of from about 1 to about 30% by weight respect to the total weight composition, (e.g., from 5 to 20% by weight). Carbohydrates may be selected from the group consisting of: fructo-oligosaccharides (or FOS), short-chain fructo-ol igosaccharides, inulin, isomalt-oligosaccharides, pectins, xylo-oligosaccharides (or XOS), chitosan-oligosaccharides (or COS), human milk oligosaccharides, beta-glucans, gum arabic modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

The composition containing yeast described herein may further contain pharmaceutically acceptable excipients or carriers. Acceptable carriers or diluents for therapeutic use are well known in the pharmaceutical art. Examples of suitable carriers include, without limitation, lactose, starch, glucose, methyl cellulose, magnesium stearate, mannitol, sorbitol and the like. Examples of suitable diluents include, without limitation, ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binders, lubricants, suspending agents, coating agents, or solubilizing agents. Examples of suitable binders include, without limitation, starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include, without limitation, sodium oleate, sodium stearate, magnesium stearate, sodium benzoate, sodium acetate, sodium chloride and the like. Preservatives, stabilizers, dyes and even flavoring agents may be provided in the pharmaceutical composition. Examples of preservatives include, without limitation, sodium benzoate, sorbic acid and esters of p-hydroxybenzoic acid. Antioxidants and suspending agents may be also used.

In some embodiments, for example when the composition described herein is formulated as a probiotic, the composition contains one or more excipients. In some embodiments, the one or more excipient is fructose, lactose monohydrate, and/or colloidal anhydrous silica.

The composition disclosed herein may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In some embodiments, the food product is a fruit juice; bar; cheese; fresh fermented product; pickle; kimchi; miso; kombucha; kefir or other fermented milks; tempeh; indigenous fermented food; sauerkraut and other fermented vegetables; coffee; cocoa and other fermented food containing yeast; sourdough bread; beer; cereal; milk; powder milk; infant formula; a composition for sportsmen like energy drinks, protein solutions/powders; specialized nutrition, e.g., for elderly or infants; hospital nutrition; medical foods.

In certain embodiments, the compositions disclosed herein contain a single yeast species or strain and do not contain any other yeast species or strains. Such compositions may comprise only de minimis or biologically irrelevant amounts of other yeast or bacterial strains or species. Such compositions may be a culture that is substantially free from other species of organism.

The composition for use in accordance with the methods disclosed herein may or may not require marketing approval.

In some cases, the lyophilized yeast, e.g. yeast strain, is reconstituted prior to administration. In some cases, the reconstitution is by use of a diluent described herein.

Any of the compositions containing yeast disclosed herein may contain pharmaceutically acceptable excipients, diluents or carriers.

In some embodiments, provided herein is a pharmaceutical composition comprising: one or more yeasts or yeast strains as disclosed herein; and a pharmaceutically acceptable excipient, carrier or diluent; wherein the yeast is present at an effective amount to treat or prevent a gastrointestinal inflammation and diseases, disorders, conditions associated therewith as described herein, including symptoms thereof.

In some embodiments, the invention provides the above pharmaceutical composition, containing a carrier selected from the group consisting of lactose, starch, glucose, methyl cellulose, magnesium stearate, mannitol and sorbitol.

In some embodiments, the invention provides the above pharmaceutical composition, comprising a diluent selected from the group consisting of ethanol, glycerol and water.

In some embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In some embodiments, the invention provides the above pharmaceutical composition, further comprising at least one of a preservative, an antioxidant and a stabilizer.

In some embodiments, the invention provides the above pharmaceutical composition, comprising a preservative selected from the group consisting of sodium benzoate, sorbic acid and esters of p-hydroxybenzoic acid.

In some embodiments, the invention provides the above pharmaceutical composition, wherein said yeast, as described herein, is lyophilized.

In some embodiments, the above pharmaceutical composition, wherein when the composition is stored in a sealed container at about 4° C. or about 25° C. and the container is placed in an atmosphere having 50% relative humidity, at least 80%, 70%, 60%, 50%, 40%, 30%, 20%, or 10% of the yeast as measured in colony forming units, remains after a period of at least about 1 month, 3 months, 6 months, 1 year, 1.5 years, 2 years, 2.5 years or 3 years.

The yeast species and strains disclosed herein can be cultured using standard microbiology techniques such as those described in the Examples section or that are well known in the art.

### II. METHODS OF TREATMENT OR PREVENTION

Provided herein are methods for using or uses of the yeasts described herein and compositions containing yeasts (e.g., probiotics, pharmaceutical compositions, topicals) as described herein. Such methods and uses include methods for treating and/or preventing gastrointestinal inflammation in a subject, such as in a human. In some embodiments, the compositions described herein are used to treat and/or prevent gastrointestinal inflammation associated with a disease, disorder, or condition, e.g., as described herein. In some embodiments, the compositions described herein treat and/or prevent diseases, disorders, or conditions associated with gastrointestinal inflammation. In some embodiments, treatment and/or prevention of gastrointestinal inflammation treats and/or prevents diseases, disorders, or conditions associated with gastrointestinal inflammation.

### A. Gastrointestinal Inflammation and Associated Diseases, Disorders, and Conditions

In some aspects, the compositions described herein treat gastrointestinal inflammation. In some embodiments, the compositions described herein prevent gastrointestinal inflammation. In some embodiments, the compositions described herein reduce gastrointestinal inflammation. In some embodiments, the gastrointestinal inflammation is any inflammation that occurs in the gastrointestinal tract. For example, inflammation present in any or all components of the entire alimentary canal, from the oral cavity to the rectum, including the mouth, esophagus, stomach, small intestine, large intestine, rectum, and the anus. The composition may be any composition described in Section I.

In some embodiments, the gastrointestinal inflammation is an acute inflammation. Acute inflammation may be inflammation that occurs for a duration of at most two weeks. In some cases, the duration of acute inflammation is less than two weeks, e.g., less than 14 days. In some cases, the duration of acute inflammation is less than one week e.g., less than 7 days. In some cases, the duration of acute inflammation is between or between about 1 day to about 14 days, about 1 day to about 10 days, about 1 day to about 7 days. In some cases, the duration of acute inflammation is between or between about 5 days and about 14 days, about 5 days and about 10 days, about 5 days and about 7 days. In some embodiments, the acute inflammation is acute enteritis or acute colitis. In some embodiments, the acute inflammation is an infection, e.g., caused by a pathogen. In some embodiments, treatment with a composition described herein reduces the duration of acute inflammation. In some embodiments, the duration of acute inflammation is reduced by or by about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, of the duration of acute inflammation that proceeds without treatment with a composition described herein.

In some embodiments, the gastrointestinal inflammation is subacute inflammation. Subacute inflammation may be inflammation that occurs for duration of greater than two weeks but no longer than 6 weeks. In some cases, the duration of subacute inflammation is between or between about 2 weeks to about 6 weeks, about 3 weeks to about 6 weeks, about 4 weeks to about 6 weeks, about 5 weeks to about 6 weeks, about 2 weeks to about 5 weeks, about 2 weeks to about 4 weeks, or about 2 weeks to about 3 weeks. In some cases, the duration of subacute inflammation is or is about 2 weeks, 3 weeks, 4 weeks, 5 weeks, or 6 weeks. In some embodiments, treatment with a composition described herein reduces the duration of subacute inflammation. In some embodiments, the duration of subacute inflammation is reduced by or by about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, of the duration of subacute inflammation that proceeds without treatment with a composition described herein.

In some embodiments, the gastrointestinal inflammation is chronic inflammation. Chronic inflammation may be inflammation that occurs for a duration of at least two months. In some cases, the duration of chronic inflammation is greater than two months. In some cases, the duration of chronic inflammation is, is at least, or is about 3, 4, 5, 6, 7, 8, 9, 10, or 11 months. In some cases, the duration of chronic inflammation is, is at least, or is about 1, 2, 3, 4, 5, or 6 years.

In some cases, the duration of chronic inflammation is indefinite. In some cases, the chronic inflammation is present throughout the natural life of a subject. In some embodiments, the chronic inflammation is an IBD, e.g., UC, CD, IC. In some embodiments, the chronic inflammation is IBS.

In some embodiments, the chronic inflammation is multiple sclerosis (MS). In some embodiments, the chronic inflammation is asthma. In some embodiments, treatment with a composition described herein reduces the duration of chronic inflammation. In some embodiments, the duration of chronic inflammation is reduced by or by about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, of the duration of chronic inflammation that proceeds without treatment with a composition described herein.

In some embodiments, the gastrointestinal inflammation, e.g., acute, subacute, or chronic inflammation, is recurrent. For example, the inflammation may cease for a period of time and then recur. In some embodiments, chronic inflammation includes periods of remission, which can be followed by a recurrence of inflammation. In some embodiments, treatment with a composition described herein prevents recurrence or relapse. In some embodiments, treatment with a composition described herein promotes remission. In some embodiments, remission lasting for at least 1, 2, 3, 4, 5, 6, or 12 months is promoted by treatment with a composition described herein. In some embodiments, remission lasting for at least 1, 2, 3, 4, 5 or more years is promoted by treatment with a composition described herein. In some embodiments, remission for the remaining life of the subject is promoted by a composition described herein, e.g., as described in Section I.

In some embodiments, the gastrointestinal inflammation arises from pathogens. In some embodiments, the gastrointestinal inflammation arises from infection by a pathogen. In some embodiments, the pathogen is a virus, fungus, or bacteria.

In some embodiments, the gastrointestinal inflammation arises from cellular or tissue damage. In some embodiments, the cellular or tissue damage occurs as a result of an autoimmune disease.

In some embodiments, the autoimmune is disease an autoimmune disease described herein. In some embodiments, the cellular or tissue damage occurs in response to a treatment, such as a cancer treatment. In some embodiments, the cancer treatment is chemotherapy, radiation therapy, or immunotherapy. In some embodiments, the treatment is any treatment described herein. In some embodiments, the cellular or tissue damage occurs as a result of diet, for example a poor diet or changes in diet.

In some embodiments, the gastrointestinal inflammation arises as a result of dysbiosis. In some embodiments, dysbiosis is or includes microbial imbalance, impaired or damaged microbiota, and/or microbial maladaptation. In some embodiments, dysbiosis causes local inflammation, e.g., gastrointestinal inflammation. In some embodiments, dysbiosis causes both local inflammation, e.g., gastrointestinal inflammation, and systemic inflammation. In some cases, dysbiosis results in systemic inflammation by promoting or facilitating the entry of endotoxins (e.g., lipopolysaccharide) into circulation, for example, into the blood stream. In some embodiments, dysbiosis is caused by the natural ageing process. In some embodiments, dysbiosis is caused by antibiotic treatment or the misuse of antibiotics. In some embodiments, dysbiosis is caused by poor diet or changes in diet. In some embodiments, dysbiosis is caused by infection. In some embodiments, dysbiosis is caused by stress or anxiety.

In some embodiments, the gastrointestinal inflammation is associated with a disease, disorder, or conditions, for example as described herein. In some embodiments, the gastrointestinal inflammation contributes to the pathogenesis of a disease, disorder, or condition and respective symptoms. In some cases, the gastrointestinal inflammation is a symptom of a disease, disorder, or condition. In some cases, treating the gastrointestinal inflammation treats the associated disease, disorder, or condition.

In some embodiments, the compositions described herein treat symptoms of gastrointestinal inflammation. In some embodiments, the compositions described herein prevent symptoms of gastrointestinal inflammation. In some embodiments, the symptoms of gastrointestinal inflammation include diarrhea, nausea, vomiting, abdominal pain, fatigue, headache, fever, and body aches. In some embodiments, one or more symptoms are reduced following treatment with a composition described herein.

In some embodiments, the gastrointestinal inflammation is associated with dysbiosis. In some embodiments, the compositions provided herein are used to treat gastrointestinal inflammation associated with dysbiosis. In some embodiments, the compositions provided herein are used to prevent gastrointestinal inflammation associated with dysbiosis. In some embodiments, the compositions provided herein are used to treat dysbiosis. In some embodiments, the compositions provided herein are used to prevent dysbiosis. In some cases, the dysbiosis is a microbial imbalance of the gastrointestinal tract, impaired or damaged microbiota of the gastrointestinal tract, or a microbial maladaptation in or to the gastrointestinal tract and/or the microbial-host ecosystem. In some embodiments, the compositions provided herein are used to reduce or prevent one or more symptoms of dysbiosis. In some embodiments, symptoms of dysbiosis include halitosis, upset stomach, gastrointestinal inflammation, upset stomach, nausea, constipation, diarrhea, difficulty urinating, vaginal or rectal itching, bloating, chest pain, rash or redness, fatigue, trouble thinking or concentrating, anxiety, and depression. Prevention or reduction in one or more symptoms, for example as described above, can be determined by any means known in the art.

In some embodiments, the gastrointestinal inflammation is associated with an IBD. In some embodiments, the compositions described herein can treat or prevent gastrointestinal inflammation associated with IBD. In some embodiments, the compositions described herein reduce or prevent symptoms of IBD. In some embodiments, symptoms of IBD include diarrhea, fever and fatigue, abdominal pain and cramping, blood in stool, reduced appetite, and unintended weight loss. In some embodiments, the symptoms of IBD are common to UC, CD, and IC.

In some embodiments, the gastrointestinal inflammation is associated with UC. In some embodiments, the compositions provided herein are used to treat gastrointestinal inflammation associated with UC. In some embodiments, the compositions provided herein are used to prevent gastrointestinal inflammation associated with UC. In some embodiments, the compositions provided herein are used to treat UC. In some embodiments, the compositions provided herein are used to prevent UC. In some cases, UC affects the innermost lining of the large intestine (colon) and rectum. In some cases, subjects with UC may experience periods of remission (e.g., greater than 1, 2, 3, 4, 5, or 6 months or at least about 1, 2, 3, 4 or more years). In some embodiments, the compositions provided herein treat and/or prevent symptoms of UC. In some cases, UC symptoms include chronic inflammation and/or ulcers in the digestive tract. In some embodiments, symptoms of UC develop overtime. In some embodiments, UC symptoms include changes in bowel movements, including diarrhea with blood or pus, urgency, and/or the inability to defecate despite urgency. In some embodiments, symptoms of UC include rectal bleeding, weight loss, fatigue, fever, and in children a failure to grow. In some embodiments, symptoms are mild to moderate in intensity.

In some cases, the UC to be treated may be a subtype of UC. In some embodiments, the UC is ulcerative proctitis. In some embodiments, ulcerative proctitis is confined to the area closest to the anus, such as the rectum. In some cases, symptoms of ulcerative proctitis include rectal bleeding. In some embodiments, the UC is proctosigmoiditis. In some cases, proctosigmoiditis affects the rectum and sigmoid colon. In some cases, symptoms of proctosigmoiditis include bloody diarrhea, abdominal cramps and pain, and an inability to move the bowels despite urgency (tenesmus). In some cases, the UC is left-sided colitis. In some embodiments, left-sided colitis includes inflammation extending from the rectum to the sigmoid colon and descending colon. In some cases, symptoms of left-sided colitis include bloody diarrhea, abdominal pain and cramping on the left side, and unintended weight loss. In some cases, the UC is pancolitis. In some embodiments, pancolitis affects the entire colon. In some cases, symptoms of pancolitis include bouts of bloody diarrhea, abdominal cramps and pain, fatigue, and significant weight loss. In some cases, the UC is acute severe UC. In some embodiments, acute severe UC is a rare form of UC that affects the entire colon. In some cases, symptoms of acute severe UC include severe pain, profuse diarrhea, bleeding, fever, and an inability to eat.

In some embodiments, symptoms of UC are or include any symptoms described herein.

In some embodiments, the gastrointestinal inflammation is associated with CD. In some embodiments, the compositions provided herein are used to treat gastrointestinal inflammation associated with CD. In some embodiments, the compositions provided herein are used to prevent gastrointestinal inflammation associated with CD. In some embodiments, the compositions provided herein are used to treat CD. In some embodiments, the compositions provided herein are used to prevent CD. In some cases, CD includes inflammation of any or all of the gastrointestinal tract, from mouth to anus. In some cases, CD is a chronic inflammatory bowel disease that affects the lining of the digestive tract. In some embodiments, the compositions provided herein are used to treat and/or prevent symptoms of CD. In some embodiments, symptoms of CD include changes in bowel movements, such as persistent diarrhea, urgency, sensation of incomplete evacuation, and/or constipation. In some embodiments, symptoms of CD include abdominal cramps and pain. In some embodiments, symptoms of CD include rectal bleeding. In some embodiments, the symptoms of CD vary between subjects. In some embodiments, symptoms of CD include the development of complications, such as fissures, fistula, and strictures. In some embodiments, systemic symptoms of CD include redness or pain in eyes, changes in vision, mouth sores, swollen and/or painful joints, skin complications (e.g., rashes, bumps, sores), fever, loss of appetite, weight loss, anemia, fatigue, night sweats, loss of normal menstrual cycle, osteoporosis, kidney stones, and liver complications (e.g., primary sclerosing cholangitis, cirrhosis).

In some embodiments, symptoms of CD are or include any symptoms described herein.

In some embodiments, the gastrointestinal inflammation is associated with IC. In some embodiments, the compositions provided herein are used to treat gastrointestinal inflammation associated with IC. In some embodiments, the compositions provided herein are used to prevent gastrointestinal inflammation associated with IC. In some embodiments, the compositions provided herein are used to treat IC. In some embodiments, the compositions provided herein are used to prevent IC. In some embodiments, the compositions provided herein are used to treat and/or prevent symptoms of IC. In some embodiments, subjects having IC present with symptoms of both UC and CD, for example symptoms as described above. In some embodiments, the symptoms of IC include, but are not limited to, abdominal pain and cramping, persistent diarrhea, blood in stool, bleeding from the rectum, unexpected or unintended weight loss, reduced appetite, fever, fatigue, and changes in bowel movement patterns including an urgent need to evacuate or a feeling of incomplete evacuation. In some embodiments, subjects having IC may be definitely diagnosed with either UC or CD.

In some embodiments, symptoms of IC are or include any symptoms described herein.

In some embodiments, treatment with the compositions described herein may reduce weight loss, blood in stool, diarrhea, or other disease symptoms of an IBD, e.g., UC, CD, IC, for example any symptom described above. Reduction in one or more symptoms can be determined by any means known in the art.

In some embodiments, treatment with the compositions described herein is prophylactic and may prevent or reduce weight loss, blood in stool, diarrhea, or other disease symptoms of an IBD, e.g., UC, CD, IC, for example any symptom described above. Prevention or reduction in one or more symptoms, for example as described above, can be determined by any means known in the art.

In some embodiments, treatment with the compositions described herein promotes remission of IBD, e.g., UC, CD, IC. In some embodiments, remission is or includes clinical remission. In some embodiments, clinical remission is when the subject does not have disease symptoms, e.g., as described herein. In some embodiments, remission is or includes biochemical remission. In some embodiments, biochemical remission is when blood and/or stool analyses do not reveal hallmarks of IBD, e.g., in blood: low red blood cell count; low levels of iron (e.g., low levels of ferritin and/or transferrin), serum albumin, folate, vitamin D, and vitamin B12, the presence of inflammatory markers (e.g., c-reactive protein (CRP)), presence of antibodies (e.g., pANCA, ASCA, CBir1, and OmpC), a high erythrocyte sedimentation rate (ESR) in blood; for stool: presence of blood. The term “low level” refers to a level that is below a level observed in a healthy subject, for example, 10%, 20%, 30%, 40%, 50%, 60%, 70%, 80%, 90%, or more below a level observed in a healthy subject not having IBD or not suspected of having IBD. In some embodiments, the remission is or includes endoscopic remission. In some embodiments, endoscopic remission is the absence of inflammation when assessed by a colonoscopy or sigmoidoscopy. In some embodiments, the remission is or includes histologic remission. In some embodiments, histologic remission is when no inflammation is seen in a biopsy, e.g., biopsy of intestinal tissue. In some embodiments, remission is or includes one or more or all types of remission described herein. In some embodiments, remission is clinical remission. In some embodiments, remission lasts for a period of at least one month, 2 months, 3 months, 4 months, 5 months, 6 months, a year, 2 years, 3 years, 4 years, 5 years or more. In some embodiments, remission lasts for an indefinite period of time or for the natural life of the subject.

In some embodiments, the gastrointestinal inflammation is associated with IBS. In some embodiments, the compositions provided herein are used to treat gastrointestinal inflammation associated with IBS. In some embodiments, the compositions provided herein are used to prevent gastrointestinal inflammation associated with IBS. In some embodiments, the compositions provided herein are used to treat IBS. In some embodiments, the compositions provided herein are used to prevent IBS. IBS is a chronic disorder affecting the large intestine requiring long term management. In some embodiments, the compositions provided herein are used to reduce or prevent one or more symptoms of IBS. In some embodiments, symptoms of IBS include cramping, abdominal pain, bloating, gas, diarrhea or constipation or both, weight loss, rectal bleeding, iron deficiency, vomiting, and difficulty swallowing. Prevention or reduction in one or more symptoms, for example as described above, can be determined by any means known in the art.

In some embodiments, the gastrointestinal inflammation is associated with a cancer. In some embodiments, the compositions provided herein are used to treat gastrointestinal inflammation associated with a cancer. In some embodiments, the compositions provided herein are used to prevent gastrointestinal inflammation associated with a cancer. In some embodiments, the compositions provided herein are used to treat a cancer. In some embodiments, the compositions provided herein are used to prevent a cancer. In some embodiments, the cancer is a cancer of a component of the gastrointestinal tract. In some embodiments, the cancer is associated with chronic inflammation, e.g., chronic gastrointestinal inflammation. In some embodiments, the cancer is colon cancer. In some embodiments, the compositions described herein reduce or prevent symptoms of colon cancer. In some embodiments, symptoms of colon cancer include diarrhea, constipation, or a change in the consistency of stool; rectal bleeding or blood in stool; persistent abdominal discomfort, such as cramps, gas or pain; a sensation of incomplete bowel evacuation, weakness or fatigue, and unexplained weight loss. In some embodiments, the cancer is stomach cancer. In some embodiments, the compositions described herein reduce or prevent one or more symptoms of stomach cancer. In some embodiments, symptoms of stomach cancer include difficulty swallowing, feeling bloated after eating, feeling full after eating small amounts of food, heartburn, indigestion, nausea, stomach pain, unintentional weight loss, and vomiting.

Prevention or reduction in one or more symptoms, for example as described above, can be determined by any means known in the art.

In some embodiments, the gastrointestinal inflammation is associated with a chemotherapy (e.g., chemotherapy-induced gastrointestinal inflammation). In some embodiments, the compositions provided herein are used to treat gastrointestinal inflammation associated with a chemotherapy.

In some embodiments, the compositions provided herein are used to prevent gastrointestinal inflammation associated with a chemotherapy. In some embodiments, the compositions provided herein are used to treat a chemotherapy-induced gastrointestinal inflammation. In some embodiments, the compositions provided herein are used to prevent a chemotherapy-induced gastrointestinal inflammation. In some embodiments, the chemotherapy-induced gastrointestinal inflammation is a result of cytotoxic cancer chemotherapy. In some embodiments, the chemotherapy-induced gastrointestinal inflammation is mucositis. Mucositis refers to ulceration and damage of mucous membranes throughout the gastrointestinal tract following chemotherapy or radiation therapy. In some cases, mucositis results in unplanned treatment interruptions, dosage reduction, or premature cessation of cancer treatment, which can have devastating health consequences for the subject. In some embodiments, the compositions described herein reduce or prevent one or more symptoms of chemotherapy-induced gastrointestinal inflammation, e.g., mucositis. In some embodiments, symptoms of chemotherapy-induced gastrointestinal inflammation, e.g., mucositis, include abdominal pain, abdominal cramping, ulceration, flatulence, nausea, vomiting, and diarrhea. Prevention or reduction in one or more symptoms, for example as described above, can be determined by any means known in the art.

In some embodiments, the gastrointestinal inflammation is associated with a radiation therapy (e.g., radiation-induced gastrointestinal inflammation). In some embodiments, the compositions provided herein are used to treat gastrointestinal inflammation associated with a radiation therapy.

In some embodiments, the compositions provided herein are used to prevent gastrointestinal inflammation associated with a radiation therapy. In some embodiments, the compositions provided herein are used to treat a radiation-induced gastrointestinal inflammation. In some embodiments, the compositions provided herein are used to prevent a radiation-induced gastrointestinal inflammation. In some embodiments, the radiation-induced gastrointestinal inflammation is a result of cytotoxic cancer radiation therapy (also referred to as radiotherapy). In some embodiments, the radiation-induced gastrointestinal inflammation is mucositis as described above. In some embodiments, the radiation-induced gastrointestinal inflammation is radiation enteritis. In some embodiments, the radiation enteritis is acute. In some embodiments, the radiation enteritis is chronic. In some embodiments, the compositions described herein reduce or prevent one or more symptoms of radiation-induced gastrointestinal inflammation, e.g., radiation enteritis. In some embodiments, symptoms of radiation-induced gastrointestinal inflammation, e.g., radiation enteritis, include abdominal pain, bloating, nausea, fecal urgency, diarrhea, and rectal bleeding. In some embodiments, the compositions described herein reduce or prevent one or more symptoms of radiation-induced gastrointestinal inflammation, e.g., mucositis. In some embodiments, symptoms of radiation-induced gastrointestinal inflammation, e.g., mucositis, include abdominal pain, abdominal cramping, ulceration, flatulence, nausea, vomiting, and diarrhea. Prevention or reduction in one or more symptoms, for example as described above, can be determined by any means known in the art.

In some embodiments, the gastrointestinal inflammation is associated with an immunotherapy (e.g., immunotherapy-induced gastrointestinal inflammation). In some embodiments, the compositions provided herein are used to treat gastrointestinal inflammation associated with an immunotherapy. In some embodiments, the compositions provided herein are used to prevent gastrointestinal inflammation associated with an immunotherapy. In some embodiments, the compositions provided herein are used to treat an immunotherapy-induced gastrointestinal inflammation. In some embodiments, the compositions provided herein are used to prevent an immunotherapy-induced gastrointestinal inflammation. In some embodiments, the immunotherapy-induced gastrointestinal inflammation is a result of cancer immune checkpoint inhibitor therapy. Immune checkpoint inhibitory therapy functions to block checkpoint proteins, e.g., PD-1, PD-L1, CTLA-4, from engaging with cognate binding partners on T cells or tumor cells, thereby allowing T cells to kill cancer cells. In some embodiments, the immunotherapy-induced gastrointestinal inflammation is immune-mediated colitis. In some embodiments, the composition described herein reduce or prevent one or more symptoms of immunotherapy-induced gastrointestinal inflammation, e.g., immune-mediated colitis. In some embodiments, symptoms of immunotherapy-induced gastrointestinal inflammation, e.g., immune-mediated colitis, include rectal bleeding, abdominal pain, diarrhea, and chronic anemia. In some embodiments, symptoms of immunotherapy-induced gastrointestinal inflammation are the same as those for IBD as described above. In some embodiments, symptoms of immunotherapy-induced gastrointestinal inflammation are the same as those for UC and subtypes of UC as described above. Prevention or reduction in one or more symptoms, for example as described above, can be determined by any means known in the art.

In some embodiments, the compositions provided herein are used to treat gastrointestinal inflammation associated with bacterial infection. In some embodiments, the compositions provided herein are used to prevent gastrointestinal inflammation associated with bacterial infection. In some embodiments, the compositions provided herein are used to treat a bacterial infection. In some embodiments, the compositions provided herein are used to prevent a bacterial infection. In some embodiments, the bacterial infection causes bacterial gastroenteritis. In some embodiments, the bacterial infection is caused by Yersinia (e.g., Y. enterocolitica), Staphylococcus (e.g., Staphylococcus aureus), Salmonella, Campylobacter(e.g., Campylobacter pylori), Clostridium difficile, or E. coli. In some embodiments, the compositions described herein reduce or prevent one or more symptoms of a bacterial infection. In some embodiments, symptoms include loss of appetite, nausea, vomiting, diarrhea, abdominal pain, abdominal cramps, blood in stool, and fever. Prevention or reduction in one or more symptoms, for example as described above, can be determined by any means known in the art.

In some embodiments, the compositions provided herein are used to treat gastrointestinal inflammation associated with a fungal infection. In some embodiments, the compositions provided herein are used to prevent gastrointestinal inflammation associated with a fungal infection. In some embodiments, the compositions provided herein are used to treat a fungal infection. In some embodiments, the compositions provided herein are used to prevent a fungal infection. In some embodiments, the fungal infection causes gastritis. In some embodiments, the fungal infection is associated with IBD. In some embodiments, the fungal infection is caused by Candida albicans or Histoplasma. In some embodiments, the compositions described herein reduce or prevent one or more symptoms of fungal infection. In some embodiments, symptoms of fungal infection resulting in gastritis include shortness of breath, chest pain, vomit that contains blood, severe stomach pain, foul-smelling bowel movements, rapid heartbeat, excessive sweating, abdominal pain, fever, yellow or green vomit, black or bloody stool, and dizziness or fainting. In some embodiments, symptoms of fungal infection include symptoms of IBD, including symptoms of IBD, UC, CD, and IC described above. Prevention or reduction in one or more symptoms, for example as described above, can be determined by any means known in the art.

In some embodiments, the compositions provided herein are used to treat gastrointestinal inflammation associated with a viral infection. In some embodiments, the compositions provided herein are used to prevent gastrointestinal inflammation associated with a viral infection. In some embodiments, the compositions provided herein are used to treat a viral infection. In some embodiments, the compositions provided herein are used to prevent a viral infection. In some embodiments, the viral infection causes gastroenteritis. In some embodiments, the viral infection is associated with IBD. In some embodiments, the viral infection is caused by rotavirus, norovirus, or adenovirus. In some embodiments, the compositions described herein reduce or prevent one or more symptoms of a viral infection, e.g., gastroenteritis. In some embodiments, symptoms of the viral infection, e.g., gastroenteritis, include nausea, vomiting, diarrhea, headache, fever, chills, and abdominal pain. In some embodiments, symptoms of the viral infection include symptoms of IBD, including symptoms of IBD, UC, CD, and IC described above. Prevention or reduction in one or more symptoms, for example as described above, can be determined by any means known in the art.

In some embodiments, the compositions provided herein are used to treat gastrointestinal inflammation associated with antibiotic use. In some embodiments, the compositions provided herein are used to prevent gastrointestinal inflammation associated with antibiotic use. In some embodiments, antibiotic use is misuse. Antibiotic misuse may include using antibiotics that are inappropriate for treatment (e.g., antibiotic treatment for a non-bacterial infection) and/or using antibiotics for shorter or longer periods than prescribed, e.g., by a medical professional. In some embodiments, antibiotic use is associated with an increased risk of IBD, such as UC and CD. In some embodiments, the compositions described herein reduce or prevent one or more symptoms of gastrointestinal inflammation associated with antibiotic use. In some embodiments, symptoms of gastrointestinal inflammation associated with antibiotic use include symptoms of IBD, including symptoms of IBD, UC, CD, and IC described above. Prevention or reduction in one or more symptoms, for example as described above, can be determined by any means known in the art.

In some embodiments, the compositions provided herein are used to treat gastrointestinal inflammation associated with obesity. In some embodiments, the obesity is class I obesity, class II obesity, class Ill obesity and pre-obesity (e.g., being “over-weight”) as defined by the World Health Organization. In some embodiments, the composition provided herein are used to prevent gastrointestinal inflammation associated with obesity. In some embodiments, obesity is associated with chronic inflammation. In some embodiments, the chronic inflammation is chronic gastrointestinal inflammation. In some embodiments, the compositions provided herein are used to treat obesity. In some embodiments, the compositions provided herein are used to prevent obesity. In some embodiments, obesity is associated with dysbiosis. In some embodiments, obesity is associated with metabolic endotoxaemia. Metabolic endotoxaemia refers to the leakage of gastrointestinal microbiota-derived molecules, such as lipopolysaccharide (LPS), out of the gastrointestinal environment that can engage pro-inflammatory pathways. In some embodiments, the compositions described herein reduce or prevent one or more symptoms of obesity. In some embodiments, symptoms of obesity include waist circumference, weight, and body mass index.

In some embodiments, the compositions described herein reduce or prevent one or more complications associated with obesity. In some embodiments, complications include heart disease and stroke, type 2 diabetes, cancers (e.g., uterus, cervix, endometrium, ovary, breast, colon, rectum, esophagus, liver, gallbladder, pancreas, kidney or prostate cancer), heartburn, gallbladder disease, liver disease, infertility and irregular periods, erectile dysfunction, sleep apnea, and osteoarthritis. Prevention or reduction in one or more symptoms or complications, for example as described above, can be determined by any means known in the art.

In some embodiments, the compositions provided herein are used to treat gastrointestinal inflammation associated with type 2 diabetes. In some embodiments, the compositions provided herein are used to prevent gastrointestinal inflammation associated with type 2 diabetes. In some embodiments, the compositions provided herein are used to treat type 2 diabetes. In some embodiments, the compositions provided herein are used to prevent type 2 diabetes. Type 2 diabetes develops when either there is impaired insulin production by the pancreas in the setting of insulin resistance in the tissues and organs in the body. In some embodiments, type 2 diabetes is associated with obesity. In some embodiments, the compositions described herein reduce or prevent one or more symptoms of type 2 diabetes. In some embodiments, symptoms of type 2 diabetes include increased thirst, frequent urination, increased hunger, unintended weight loss, fatigue, blurred vision, slow-healing wounds or sores, frequent infections, numbness or tingling in the hands or feet, areas of darkened skin typically around the armpits and neck. In some embodiments, the compositions described herein reduce or prevent one or more complications associated with type 2 diabetes. In some embodiments, complications include heart and blood vessel disease, nerve damage, kidney disease, glaucoma, cataracts, blindness, bacterial and fungal skin infections, hearing impairment, sleep apnea, and dementia. Prevention or reduction in one or more symptoms or complications, for example as described above, can be determined by any means known in the art.

In some embodiments, the compositions provided herein are used to treat gastrointestinal inflammation associated with metabolic syndrome. In some embodiments, the compositions provided herein are used to prevent gastrointestinal inflammation associated with metabolic syndrome. In some embodiments, the compositions provided herein are used to treat metabolic syndrome. In some embodiments, the compositions provided herein are used to prevent metabolic syndrome. Metabolic syndrome refers to a cluster of conditions that increase the risk of heart disease, diabetes (e.g., type 2 diabetes), and stoke. In some embodiments, conditions of metabolic syndrome include one or more of high blood pressure, high blood sugar, excess body fat around the waist, and abnormal cholesterol levels. In some embodiments, the metabolic syndrome increases a subject's chances of heart attack and stroke. In some embodiments, metabolic syndrome is associated with obesity. In some embodiments, metabolic syndrome is associated with physical inactivity. In some embodiments, the compositions described herein reduce or prevent one or more symptoms of metabolic syndrome. The cluster of conditions constituting metabolic syndrome may be associated with few or no obvious symptoms. In some embodiments, a large waist circumference is a symptom of metabolic syndrome. In some cases, for example if the subject has high blood sugar, metabolic syndrome has symptoms of diabetes, such as symptoms of type 2 diabetes as described above. In some embodiments, the compositions described herein reduce or prevent one or more complications associated with metabolic syndrome. In some embodiments, complications include type 2 diabetes and heart and blood vessel disease. Prevention or reduction in one or more symptoms or complications, for example as described above, can be determined by any means known in the art.

In some embodiments, the compositions provided herein are used to treat gastrointestinal inflammation associated with asthma. In some embodiments, the compositions provided herein are used to prevent gastrointestinal inflammation associated with asthma. In some embodiments, the compositions provided herein are used to treat asthma. In some embodiments, the compositions provided herein are used to prevent asthma. Asthma is a chronic inflammatory disorder of the lower airs. Research has suggested a relationship between the gastrointestinal dysbiosis and asthma. In some embodiments, asthma is associated with a reduced level of Lachnospira and an increased level Clostridium spp. In some embodiments, increased levels of Clostridium spp are indicative of a risk of developing asthma. Increased and decreased levels are made with reference to healthy individual not having asthma or not at risk of developing asthma. In some embodiments, increased and decreased levels include, independently, differences of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to levels in healthy individuals not having asthma or not at risk of developing asthma. In some embodiments, the compositions described herein reduce or prevent one or more symptoms of asthma. In some embodiments, symptoms of asthma include shortness of breath, chest tightness or pain, wheezing, trouble sleeping, and coughing or wheezing attacks that may be worsened by a respiratory virus. Prevention or reduction in one or more symptoms, for example as described above, can be determined by any means known in the art.

In some embodiments, the compositions provided herein are used to treat gastrointestinal inflammation associated with atherosclerosis. In some embodiments, the compositions provided herein are used to prevent gastrointestinal inflammation associated with atherosclerosis. In some embodiments, the compositions provided herein are used to treat atherosclerosis. In some embodiments, the compositions provided herein are used to prevent atherosclerosis. Atherosclerosis refers to the build up of fats, cholesterol, and other substances that form plaques on artery walls and restrict blood flow. Inflammation, for example systemic inflammation associated with gastrointestinal inflammation, e.g., as a result of dysbiosis, has been associated with atherogenesis. In some embodiments, the compositions described herein reduce or prevent one or more symptoms of atherosclerosis. In some embodiments, symptoms of atherosclerosis include chest pain (angina), numbness or weakness in arms or legs, difficulty speaking, slurred speech, temporary loss of vision in one eye, drooping facial muscles, transient ischemic attack, peripheral artery disease, leg pain during walking (claudication), high blood pressure, and kidney failure. In some embodiments, the compositions described herein reduce or prevent one or more complications associated with atherosclerosis. In some embodiments, complications include coronary artery disease, carotid artery disease, peripheral artery disease, aneurysms, and chronic kidney disease. Prevention or reduction in one or more symptoms or complications, for example as described above, can be determined by any means known in the art.

In some embodiments, the compositions provided herein are used to treat gastrointestinal inflammation associated with non-alcoholic fatty liver disease. In some embodiments, the compositions provided herein are used to prevent gastrointestinal inflammation associated with non-alcoholic fatty liver disease. In some embodiments, the compositions provided herein are used to treat non-alcoholic fatty liver disease. In some embodiments, the compositions provided herein are used to prevent non-alcoholic fatty liver disease. Non-alcoholic fatty liver disease refers to a arrange of liver conditions characterized by the buildup of fat in liver cells that is not caused by alcohol consumption. Inflammation, for example as a result of gastrointestinal dysbiosis, is considered a factor in the pathogenesis and progression of non-alcoholic fatty liver disease. In some embodiments, the non-alcoholic fatty liver disease is nonalcoholic steatohepatitis or cirrhosis. In some embodiments, the compositions described herein reduce or prevent one or more symptoms of non-alcoholic fatty liver disease. In some embodiments, symptoms of non-alcoholic fatty liver disease include fatigue and pain or discomfort in the upper right abdomen. In some embodiments, when the non-alcoholic fatty liver disease is nonalcoholic steatohepatitis or cirrhosis the symptoms include abdominal swelling, enlarged blood vessels beneath the skin's surface, enlarged spleen, red palms, and jaundice. In some embodiments, the compositions described herein reduce or prevent one or more complications associated with non-alcoholic fatty liver disease. In some embodiments, complications include cirrhosis. Left unattended, cirrhosis may result in symptoms including fluid buildup in the abdomen, swelling of veins in the esophagus, confusion, drowsiness, slurred speech, liver cancer, and end-stage liver failure. Prevention or reduction in one or more symptoms or complications, for example as described above, can be determined by any means known in the art.

In some embodiments, the compositions provided herein are used to treat gastrointestinal inflammation associated with multiple sclerosis. In some embodiments, the compositions provided herein are used to prevent gastrointestinal inflammation associated with multiple sclerosis. In some embodiments, the compositions provided herein are used to treat multiple sclerosis. In some embodiments, the compositions provided herein are used to prevent multiple sclerosis. Multiple sclerosis is a chronic, inflammatory, demyelinating, and degenerative disease that affects the central nervous system. Inflammation, including gastrointestinal inflammation for example driven by dysbiosis and systemic inflammation resulting therefrom, is considered a factor in the pathogenesis and progression of multiple sclerosis. In some embodiments, the compositions described herein reduce or prevent one or more symptoms of multiple sclerosis. In some embodiments, symptoms of multiple sclerosis include numbness or weakness in one or more limbs, typically on one side of the body; electric-shock like sensations with neck movements; tremor; lack of coordination; unsteady gait; partial or complete vision loss, usually one eye at a time and associated with painful eye movements; prolonged double vision; blurry vision; slurred speech; fatigue; dizziness; tingling or pain in parts of the body; problems with sexual, bowel, and bladder function. In some embodiments, the multiple sclerosis includes relapse and remission cycles. In some embodiments, the compositions described herein prevent relapse. In some embodiments, the compositions described herein reduce or prevent one or more complications associated with multiple sclerosis. In some embodiments, complications include muscle stiffness or spasms; paralysis, typically in the legs; problems bladder, bowel or sexual function; mental changes as forgetfulness or mood swings; depression; and epilepsy. Prevention or reduction in one or more symptoms or complications, for example as described above, can be determined by any means known in the art.

In some embodiments, the compositions provided herein are used to treat gastrointestinal inflammation associated with the natural aging process. In some embodiments, the compositions provided herein are used to prevent gastrointestinal inflammation associated with the natural aging process. In some cases, the process of aging includes changes in the microbiome which can lead to dysbiosis and the release of pro-inflammatory cytokines. Thus, in some cases, age-related alterations in the microbiome promote gastrointestinal inflammation, which may facilitate IBD (e.g., UC, CD, IC), IBS, metabolic syndrome, diabetes mellitus types 1 and 2, and obesity. In some embodiments, the composition described herein reduce or prevent symptoms of gastrointestinal inflammation associated with the aging process, such as symptoms of dysbiosis. In some embodiments, symptoms of gastrointestinal inflammation associated with associated with aging process include symptoms of IBD, including symptoms of UC, CD, and IC described above. In some embodiments, symptoms of gastrointestinal inflammation associated with aging process include symptoms of IBS, including symptoms of IBS described above. In some embodiments, symptoms of gastrointestinal inflammation associated with aging process include symptoms of metabolic syndrome, including symptoms of metabolic syndrome described above. In some embodiments, symptoms of gastrointestinal inflammation associated with aging process include symptoms of obesity, including symptoms of obesity described above. In some embodiments, symptoms of gastrointestinal inflammation associated with aging process include symptoms of type 2 diabetes, including symptoms of type 2 diabetes described above. Prevention or reduction in one or more symptoms, for example as described above, can be determined by any means known in the art.

In some embodiments, the compositions provided herein are used to treat gastrointestinal inflammation associated with skin inflammation. In some embodiments, the compositions provided herein are used to prevent gastrointestinal inflammation associated with skin inflammation. In some embodiments, the compositions provided herein are used to treat skin inflammation. In some embodiments, the compositions provided herein are used to prevent skin inflammation. Cutaneous manifestations of gastrointestinal disorders are well-documented, and there is substantial evidence to suggest that gastrointestinal dysbiosis plays a role in the pathophysiology of a variety of inflammatory disorders of the skin. In some embodiments, the skin inflammation is acne. Acne may include symptoms such as closed plugged pores, open plugged pores, papules, pustules, nodules, and cystic lesions, typically on the face, forehead, chest upper back and shoulders. In some embodiments, the skin inflammation is atopic dermatitis. Atopic dermatitis may have symptoms such as dry skin, itching, red to brownish-gray skin patches, small raise bumps that may leak fluid and crust, and thickened, cracked, scaly, raw, sensitive, and swollen skin. In some embodiments, the skin inflammation is psoriasis or a subtype thereof including plaque psoriasis, nail psoriasis, guttate psoriasis, inverse psoriasis, pustular psoriasis, erythrodermic psoriasis, or psoriatic arthritis. Symptoms of psoriasis and subtypes thereof may include: dry raise red skin lesions covered with silvery scales that may be itchy or tender as found in plaque psoriasis; pitting abnormal nail growth and discoloration, onycholysis, and nail crumbling as found on in nail psoriasis; small, drop-shaped, scaling lesions on the trunk, arms or legs, as found in guttate psoriasis; smooth patches of red skin around the groin, buttocks, and breasts as found in inverse psoriasis; red, peeling rash covering the body as found in erythrodermic psoriasis; and swollen painful joints and nail changes as found in psoriatic arthritis. In some embodiments, the skin inflammation is associated with an IBD, such as UC and CD. In some embodiments, the skin inflammation is erythema nodosum. Erythema nodsum symptoms may include tender red nodules that appear on the arms or legs. In some embodiments, the skin inflammation is puroderma gangrenosum. Puroderma gangrenosum may include symptoms such as clusters of small blisters that combine to form deep ulcers typically on the shins and ankles. In some embodiments, the skin inflammation is Sweet's syndrome. Sweet's syndrome may include symptoms such as painful skin lesions that start as small tender red or purple bumps that spread to painful clusters typically on the face, neck, or upper limbs. In some embodiments, the skin inflammation is bowel-associated dermatosis-arthritis syndrome. Bowel-associated dermatosis-arthritis syndrome may include symptoms such as small, painful bumps that may form pustules over time typically found on the upper chest and arms. In some embodiments, the skin inflammation is vitiligo. Vitiligo symptoms may include white patches of skin resulting from the death of pigment producing cells. In some embodiments, the skin inflammation is pyrodermatitis-pyostomatitis vegetans. Pyrodermatitis-pyostomatitis vegetans may include symptoms such as rashes with red pustules that may form plaques and mouth pustules. In some embodiments, the skin inflammation is leukocytoclastic vasculitis. Leukocytoclastic vasculitis may include symptoms such as small blood vessel bursting and blood pooling under the skin resulting in purpura. In some embodiments, the skin inflammation is hives. Hives may include symptoms such as red, itchy rashes. In some embodiments, the compositions described herein reduce or prevent one or more symptoms associated with a skin inflammation described herein. Prevention or reduction in one or more symptoms, for example as described above, can be determined by any means known in the art.

In some embodiments, decreases in any symptom or complication described herein include decreases of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, in expression of the symptom or complication relative to expression of the symptom or complication prior to treatment with a composition described herein. In some embodiments, decreases in any symptom or complication provided herein include decreases of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, in expression of the symptom or complication relative to a subject having said symptom or complication and not administered a composition described herein.

### B. Administration

In some embodiments, a composition described herein (such as probiotics, pharmaceutical compositions, topicals) is administered to a subject. See, e.g., Section I. In some embodiments, the subject has been diagnosed with gastrointestinal inflammation and/or a disease, disorder or condition associated with gastrointestinal inflammation. In some embodiments, the subject has symptoms of gastrointestinal inflammation and/or a disease, disorder, or condition associated therewith. In some embodiments, the subject is suspected of having or developing gastrointestinal inflammation and/or a disease, disorder, or condition associated therewith. In some embodiments, the subject has a disease, disorder, or condition as described above in Section II-A. In some embodiments, the subject has symptoms associated with gastrointestinal inflammation and/or a disease, disorder, or condition associated therewith, for example as described above in Section II-A. In some embodiments, the subject has gastrointestinal inflammation. In some embodiments, the subject has gastrointestinal inflammation and systemic inflammation, e.g., resulting from dysbiosis. In some embodiments, the subject is in remission from IBD, for example a remission as described in Section II-A.

Generally, dosages and routes of administration of the composition are determined according to the size, e.g., weight, and condition of the subject, according to standard pharmaceutical practice. For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage can be determined in light of factors related to the subject requiring treatment. Dosage and administration can be adjusted to provide sufficient levels of the active compound, e.g., yeast species or strain, or to maintain the desired effect. Factors that may be taken into account include the severity of the disease state, the general health of the subject, the age, weight, and gender of the subject, time and frequency of administration, drug combination(s), reaction sensitivities, and response to treatment.

In some embodiments, the compositions containing yeast disclosed herein are to be administered to the gastrointestinal tract in order to enable delivery to and/or partial or total colonization of the intestine with the yeast described herein. In some embodiments, the compositions of the invention are administered orally. In some embodiments, the compositions of the invention are administered topically. In some embodiments, the compositions of the inventions are administered rectally, intranasally, or via buccal or sublingual routes.

In some embodiments, the compositions disclosed herein may be administered as a foam, as a spray or a gel.

In some embodiments, the compositions disclosed herein may be administered as a suppository, such as a rectal suppository, for example in the form of a theobroma oil (cocoa butter), synthetic hard fat (e.g. suppocire, witepsol), glycero-gelatin, polyethylene glycol, or soap glycerin composition.

In some embodiments, the compositions disclosed herein are administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to tire stomach, jejunum and other suitable access ports.

In some embodiments, for example when the disease, disorder, or conditions, or a symptom of the disease, disorder, or condition is a skin inflammation, the composition described herein may be administered as a topical. In some embodiments, the topical is administered by a patch. In some embodiments, topicals may be administered according to any of those described in Section I-B.

In some embodiments, the compositions containing yeast disclosed herein (e.g., probiotics, pharmaceutical compositions, topicals) may be administered once, or they may be administered sequentially as part of a treatment regimen. In some embodiments, the compositions of the invention are to be administered daily. In some embodiments, the compositions of the invention are to be administered at least two times daily. In some embodiments, the compositions of the invention are to be administered for at least 1, 2, 3, or 4 weeks. In some embodiments, the compositions of the invention are to be administered until symptoms are reduced, e.g., as described herein. In some embodiments, the compositions of the invention are to be administered until the subject does not present with symptoms. In some embodiments, the compositions of the invention are to be administered indefinitely. In some embodiments, the compositions of the invention are to be administered throughout the natural life of a subject. In some embodiments, the compositions of the invention are to be administered intermittently, e.g., periodically, throughout the natural life of a subject.

In some embodiments, treatment with the compositions disclosed herein according to methods disclosed herein is accompanied by assessment of the subject's gut microbiota. Treatment may be repeated if delivery of and/or partial or total colonization with the yeasts, e.g., yeast strains, described herein are not achieved such that efficacy is not observed, or treatment may be ceased if delivery and/or partial or total colonization is successful, and efficacy is observed.

In some embodiments, the compositions containing yeast disclosed herein are administered as part of a combination treatment. For example, in some cases, the compositions described herein may be administered in combination with an existing pharmacological treatment, such as cyclosporin.

In some embodiments, the compositions containing yeast disclosed herein can be administered as a food product, such as a nutritional supplement.

In some embodiments, methods provided herein include administering a composition described herein in an amount that is known or predicted to achieve a therapeutic effect, e.g., treat, prevent, reduce gastrointestinal inflammation and/or diseases, disorders, and conditions associated therewith (see, e.g., Section II-A). In some embodiments, the amount of yeast administered is in an amount that reduces or is predicted to reduce a symptom as described herein (see, Section II-A) by at least or at least about 10%, 20%, 30%, 40%, 50%, 60%, 70%, 80%, 90%, or more compared to the presentation of symptoms prior to or at the initiation of treatment with a composition containing yeast as described herein. In some embodiments, treatment with a composition containing yeast described herein reduces symptoms as described in Section II-A, for a period of at least 1, 2, 3, 4, 5, 6, 7, 8, 9, 10, 11, or 12 months. In some embodiments, the period may be for between about a month to a year, a month to 6 months, or a month to 3 months.

In some embodiments, for example when the composition containing yeast promotes remission, e.g., an IBD remission or multiple sclerosis remission as described in Section II-A, the period of remission may be for at least 1, 2, 3, 4, 5, 6, 7, 8, 9, 10, 11, or 12 months. In some embodiments, the period of remission may be for between about a month to a year, a month to 6 months, or a month to 3 months.

### III. KITS

Also provided are kits including the compositions containing yeast, e.g., probiotics, pharmaceutical compositions, topicals, described herein, which may further include instructions on methods of using the composition, such as uses described herein. The kits described herein may also include other materials desirable from a commercial and user standpoint, including other buffers, diluents, filters, needles, syringes, and package inserts with instructions for performing any methods described herein.

### IV. DEPOSIT AND EXPERT SOLUTION

The following deposit was made according to the Budapest treaty on the international recognition of the deposit of microorganisms for the purposes of patent procedure.
Cyberlindnera jadinii strain (DGCC3540) deposited under accession number DSM 33763 on Jan. 19, 2021, at the DSMZ [Deutsche Sammlung von Mikroorganismen und Zellkulturen GmbH, Inhoffenstrasse 7B, D-38124 Braunschweig—Germany].
Kluyveromyces lactis strain (DGCC3550) deposited under accession number DSM 33764 on Jan. 19, 2021, at the DSMZ [Deutsche Sammlung von Mikroorganismen und Zellkulturen GmbH, Inhoffenstrasse 7B, D-38124 Braunschweig—Germany].

It is requested that the biological material shall be made available only by the issue of a sample to an expert nominated by the requester. In respect to those designations in which a European Patent is sought, a sample of the deposited microorganism will be made available until the publication of the mention of the grant of the European patent or until the date on which application has been refused or withdrawn or is deemed to be withdrawn, only by the issue of such a sample to an expert nominated by the person requesting the sample, and approved either i) by the Applicant and/or ii) by the European Patent Office, whichever applies (Rule 32 EPC)

### V. EXEMPLARY EMBODIMENTS

1. A composition comprising Cyberlindnera jadinii.
2. The composition of embodiment 1, wherein the Cyberlindnera jadinii is the strain deposited at DSMZ [Deutsche Sammlung von Mikroorganismen und Zellkulturen GmbH, Inhoffenstrasse 7B, D-38124 Braunschweig—Germany] under number DSM 33763 or a strain having all of the identifying characteristics of the Cyberlindnera jadinii strain deposited at DSMZ under number DSM 33763.
3. The composition of embodiment 1 or embodiment 2, wherein the Cyberlindnera jadinii is dried.
4. The composition of any one of embodiments 1-3, comprising an effective amount of the Cyberlindnera jadinii to treat and/or prevent gastrointestinal inflammation and/or a disease, disorder, or condition associated with gastrointestinal inflammation.
5. The composition of any one of embodiments 1-4, comprising an amount of the Cyberlindnera jadinii that is at least about 1×10⁹ CFU/g to at least about 5×10¹⁰ CFU/g of composition.
6. The composition of any one of embodiments 1-5, comprising an amount of the Cyberlindnera jadinii that is or is about 2.5×10¹⁰ CFU/g of composition.
7. A composition comprising Kluyveromyces lactis.
8. The composition of embodiment 7, wherein the Kluyveromyces lactis is the strain deposited at DSMZ [Deutsche Sammlung von Mikroorganismen und Zellkulturen GmbH, Inhoffenstrasse 7B, D-38124 Braunschweig—Germany] under number DSM 33764 or a strain having all of the identifying characteristics of the Kluyveromyces lactis strain deposited at DSMZ under number DSM 33764.
9. The composition of embodiment 7 or embodiment 8, wherein the Kluyveromyces lactis is dried.
10. The composition of any one of embodiments 7-9, comprising an effective amount of the Kluyveromyces lactis to treat and/or prevent gastrointestinal inflammation and/or a disease, disorder, or condition associated with gastrointestinal inflammation.
11. The composition of any one of embodiments 7-10, comprising an amount of the Kluyveromyces lactis that is at least about 1×10⁹ CFU/g to at least about 5×10¹⁰ CFU/g of composition.
12. The composition of any one of embodiments 7-11, comprising an amount of the Kluyveromyces lactis that is or is about 2.5×10¹⁰ CFU/g of composition.
13. The composition of any one of embodiments 1-12, wherein the composition is a probiotic.
14. The composition of any one of embodiments 1-13, wherein the composition is a pharmaceutical composition and further comprises at least one pharmaceutically acceptable carrier and/or excipient.
15. The composition of any of embodiments 1-14, wherein the composition is formulated for oral administration.
16. The composition of any one of embodiments 1-15, wherein the composition is encapsulated or coated.
17. The composition of any one of embodiments 1-16, wherein the composition is a food product, food ingredient, dietary supplement, or medicament.
18. The composition of any of embodiments 1-14, wherein the composition is formulated for topical administration.
19. The composition of any one of embodiments 4-6 and 10-18, wherein the disease, disorder, or condition is IBD, IBS, cancer, chemotherapy-induced gastrointestinal inflammation, radiation-induced gastrointestinal inflammation, immunotherapy-induced gastrointestinal inflammation, bacterial infection, viral infection, fungal infection, antibiotic use, aging, dysbiosis, obesity, type 2 diabetes, metabolic syndrome, asthma, atherosclerosis, non-alcoholic fatty liver disease, multiple sclerosis, or skin inflammation.
20. A tablet, chewable gum, capsule, granule, powder, sachet, patch, cream, or ointment comprising the composition of any one of embodiments 1-19.
21. A kit comprising:
   (a) the composition of any one of embodiments 1-19 or the tablet, chewable gum, capsule, granule, powder, sachet, patch, cream, or ointment of embodiment 20; and
   (b) written instructions for administration to a subject.
22. A method for treating and/or preventing gastrointestinal inflammation and/or a disease, disorder or condition associated with gastrointestinal inflammation, in a subject in need thereof, comprising administering an effective amount of the composition of any one of embodiments 1-19 or the tablet, chewable gum, capsule, granule, powder, sachet, patch, cream, or ointment of embodiment 20 to the subject.
23. The method of embodiment 22, wherein the disease, disorder, or condition is IBD, IBS, cancer, chemotherapy-induced gastrointestinal inflammation, radiation-induced gastrointestinal inflammation, immunotherapy-induced gastrointestinal inflammation, bacterial infection, viral infection, fungal infection, antibiotic use, aging, dysbiosis, obesity, type 2 diabetes, metabolic syndrome, asthma, atherosclerosis, non-alcoholic fatty liver disease, multiple sclerosis, or skin inflammation.
24. The method of embodiment 22 or embodiment 23, wherein the disease, disorder, or condition is IBD.
25. The method of embodiment 23 or embodiment 24, wherein the IBD is ulcerative colitis or Crohn's disease.
26. The method of embodiment 22 or embodiment 23, wherein the disease, disorder or condition is dysbiosis.
27. A composition for use in the treatment and/or prevention of gastrointestinal inflammation and/or a disease, disorder, or condition associated with gastrointestinal inflammation in a subject in need thereof, comprising the composition of any one of embodiments 1-19 or the tablet, chewable gum, capsule, granule, powder, sachet, patch, cream, or ointment of embodiment 20.
28. The composition for use of embodiment 27, wherein the disease, disorder, or condition is IBD, IBS, cancer, chemotherapy-induced gastrointestinal inflammation, radiation-induced gastrointestinal inflammation, immunotherapy-induced gastrointestinal inflammation, bacterial infection, viral infection, fungal infection, antibiotic use, aging, dysbiosis, obesity, type 2 diabetes, metabolic syndrome, asthma, atherosclerosis, non-alcoholic fatty liver disease, multiple sclerosis, or skin inflammation.
29. The composition for use of embodiment 27 or embodiment 28, wherein the disease, disorder, or condition is IBD.
30. The composition for use of embodiment 28 or embodiment 29, wherein the IBD is ulcerative colitis or Crohn's disease.
31. The composition for use of embodiment 27 or embodiment 28, wherein the disease, disorder or condition is dysbiosis.

### VI. EXAMPLES

The following examples are included for illustrative purposes only and are not intended to limit the scope of the invention.

### Example 1: Assessment of Fungal Treatment in an Animal Model of Colitis

The impact of treatment with yeast cells on inflammatory bowel disease (IBD) was evaluated in an induced mouse model of colitis.

### Materials and Methods

### Yeast Cultures

Cyberlindnera jadinii (DSM 33763), Debaryomyces hansenii, Kluyveromyces lactis (DSM 33764), Kazachstania unispora, and Pichia membranifaciens yeast cells were cultivated independently on yeast extract peptone dextrose (YEPD) agar (Sigma-Aldrich France) for 24 h to 48 h at 30° C. The preculture and culture of yeast were prepared in YEPD medium and then incubated for 24 h at 30° C. under agitation at 150 rpm.

### Mouse Model of Ulcerative Colitis

Eight-week-old mice female C57BL/6J were purchased from Janvier (France) and used one week after arrival. All experiments were performed in accordance with the ethic committee in animal experiment (COMETHEA C2EA, Jouy en Josas, France). Every experiment was repeated at least two times.

Mice were gavaged (intragastric gavage) with a suspension of yeast cells in phosphate buffered saline (vehicle) at 1×10⁷ CFU per gavage/mouse/day for 19 days.

One week after starting the fungal administration, mice were given 2% (weight/volume) Dextran Sodium Sulfate (DSS) colitis grade (MP Biomedicals, Solon, OH) dissolved in the drinking water for 7 days, followed by a recovery period (water only) of 5 days. The animals were monitored daily beginning at the onset of DSS treatment for weight loss and disease activity index (DAI). The DAI includes three parameters with a score notation from 0 to 4: weight loss, stool consistency and presence of blood in the feces (see, Table E1).

TABLE E1
Disease activity index exemplary scores

| Score | Consistency stool | Blood | Body weight |
|---|---|---|---|
| 0 | Normal | Negative (−) | <1% |
| 2 | Loose stools | + | 5-10% |
| 4 | Diarrhea | Bleeding | >15% |

### Determination of Yeast Cell Survival

Animal stool was collected over the course of the study to determine the quantity of yeast remaining after intragastric gavage. Fresh stools were weighed and suspended in phosphate-buffered saline (PBS) (GIBCO, Fischer-SCI, France) in proportion of 40 μL/mg of feces. For quantification, the dilutions of feces were plated on YEPD agar (Sigma-Aldrich France) plates supplemented with ampicillin (100 mg/mL Sigma-Aldrich France), penicillin/streptomycin (50 mg/mL GIBCO Fisher-Scientific France) and incubated at 30° C. for 24 to 48 h. Colonies were then counted, and the absolute quantities of yeast were determined according to the corresponding dilutions.

### Anti-Inflammatory Treatment in the DSS-Induced Colitis Model

For comparison with yeast treatment, a separate group of mice were administered cyclosporin beginning at the onset of treatment with DSS to induce ulcerative colitis (day 0) until sacrifice. Cyclosporin was administered IP at 100 μL/mouse (at a concentration of 25 mg/kg) and injections were performed 3 times a week.

### Statistical Analysis

Graph Pad Prism software was used for all analyses and illustrations. All results were expressed using the mean±SEM (n=10/group). The comparison between all the groups was carried out with a non-parametric one-way t-test (ANOVA). For all statistical tests, differences with a p value less than 0.05 were considered significant.

### Results

### Yeast Cell Survival

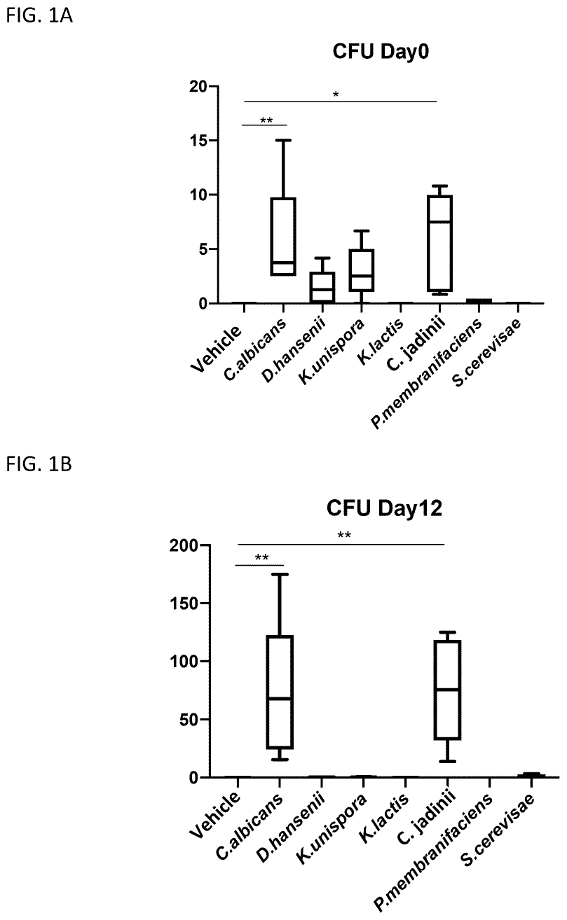

The ability of yeasts used in this study to survive in the mouse gut environment was assessed as described in the Materials and Methods above. FIGS. 1A and 1B show the levels of yeast present on day 0 (initiation of DSS treatment) and at day 12. As shown in FIGS. 1A-1B, C. jadinii showed increased survival in the mouse gut environment compared to the other tested yeast strains. C. jadinii survival at day 12 was similar to the pathogenic Candida albicans, which is known for efficient survival in the gastrointestinal tract of mice and humans.

### Weight Protection

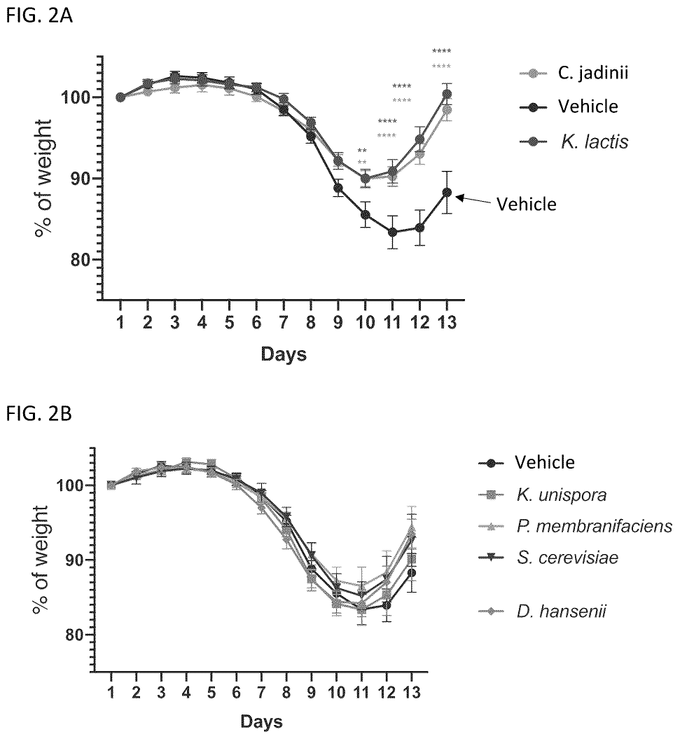

FIGS. 2A and 2B show weight loss over time in DSS-induced colitis mice treated with yeast or with buffer used for yeast cell suspension (vehicle). FIG. 2A shows that treatment with C. jadinii or K. lactis provides an unexpected protective effect on body weight (Two-way Anova: **p<0.005, ****p<0.0001). As can be seen in FIG. 2A, mice treated with C. jadinii or K. lactis demonstrated fast weight recovery, with mice reaching 100% of their initial weight by day 13.

FIG. 2B shows that mice treated with yeast species K. unispora, P. membranifaciens, S. cerevisiae, or D. hansenii had similar weights to vehicle treated mice.

### Disease Activity Index

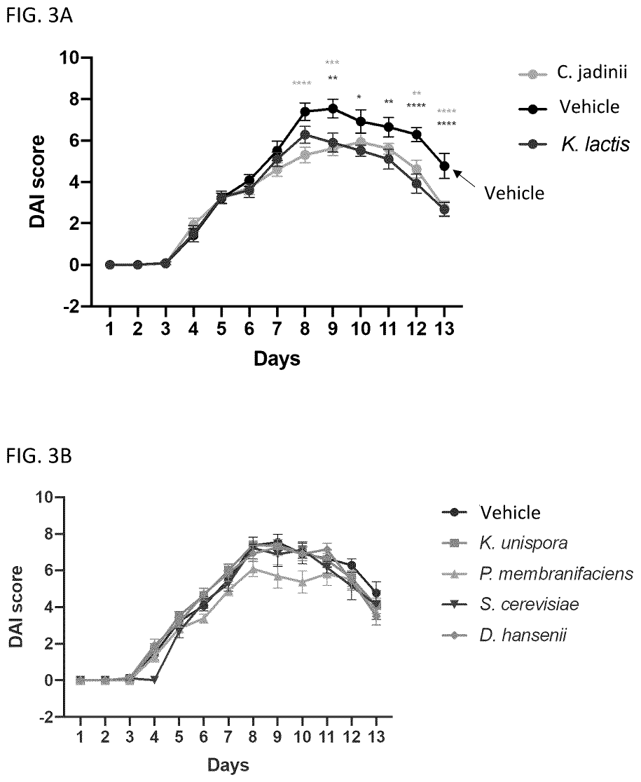

DAI is widely used to quantify the effect of DSS-induced colitis on the overall health status of the animal. The Disease Activity Index (DAI: which is a calculated scientifically recognized mathematical combination of total animal body Weight/quantity of Blood in feces & feces texture) was therefore used to assess the effects of yeast treatment on DSS-induced mouse models of ulcerative colitis. FIGS. 3A and 3B show DAI scores over time (days).

As seen in FIG. 3A, mice treated with C. jadinii or K. lactis showed lower DAI scores than mice treated with buffer alone (vehicle). As shown in FIG. 3B, mice treated with the other tested yeast strains exhibited similar DAI scores to mice who received treatment with buffer alone (vehicle).

Comparison of Yeast Treatment to Treatment with Immunosuppressants

The effects of C. jadinii or K. lactis treatment on ulcerative colitis were compared to DSS-induced colitis mice treated with a widely used immunosuppressant medication, cyclosporin (also spelled cyclosporine and ciclosporin), a calcineurin inhibitor. In common trials, mice were treated with cyclosporin as described in the Materials and Methods above.

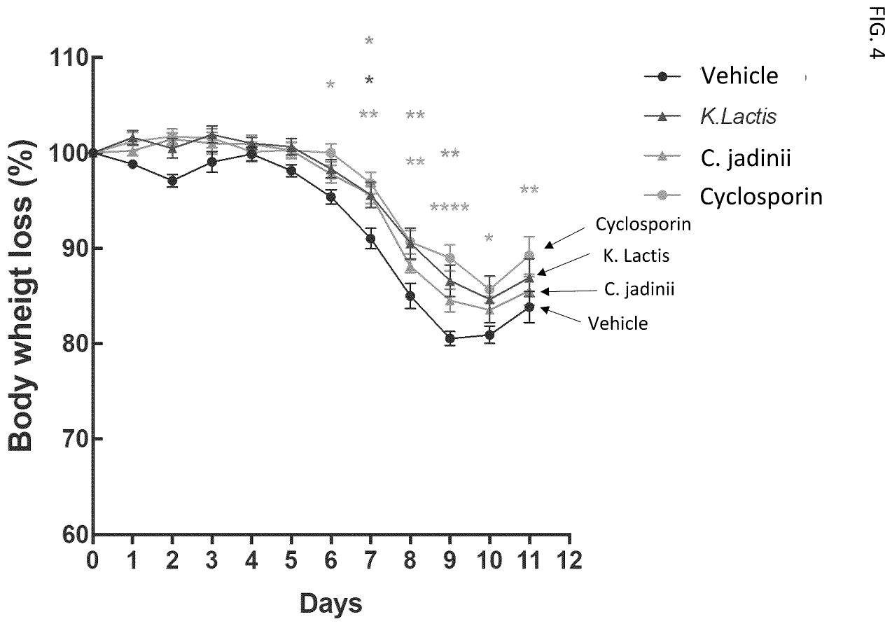

As shown in FIG. 4, treatment with the yeasts or cyclosporin resulted in similar weight protection. These results suggest that C. jadinii and K. lactis may have similar an anti-inflammatory effects as cyclosporin.

### CONCLUSIONS

These data support C. jadinii and K. lactis yeast cells in the treatment and/or prevention of inflammation. These data further support the use of C. jadinii and K. lactis in the treatment or prevention of IBD, such as ulcerative colitis. The data suggest that C. jadinii and K. lactis are useful for treating and/or preventing gastrointestinal inflammation.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. Although the invention may be described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. (canceled)

2. The method of claim 12, wherein the Cyberlindnera jadinii is the strain deposited at DSMZ [Deutsche Sammlung von Mikroorganismen und Zellkulturen GmbH, Inhoffenstrasse 7B, D-38124 Braunschweig—Germany] under number DSM 33763 or a strain having all of the identifying characteristics of the Cyberlindnera jadinii strain deposited at DSMZ under number DSM 33763.

3. The method of claim 12, wherein the Kluyveromyces lactis is the strain deposited at DSMZ [Deutsche Sammlung von Mikroorganismen und Zellkulturen GmbH, Inhoffenstrasse 7B, D-38124 Braunschweig—Germany] under number DSM 33764 or a strain having all of the identifying characteristics of the Kluyveromyces lactis strain deposited at DSMZ under number DSM 33764.

4. The method of claim 12, wherein the effective amount of the Cyberlindnera jadinii or the Kluyveromyces lactis in the composition is present at from about 1×10⁹ CFU/g to about 5×10¹⁰ CFU/g of composition.

5. The method of claim 12, wherein the composition is a probiotic.

6. The method of claim 12, wherein the composition is a pharmaceutical composition and further comprises at least one pharmaceutically acceptable carrier and/or excipient.

7. The method of claim 12, wherein the composition is formulated for oral administration or topical administration.

8. The method of claim 12, wherein the composition is a food product, food ingredient, dietary supplement, or medicament.

9. The method of claim 12, wherein the disease, disorder, or condition is IBD, IBS, cancer, chemotherapy-induced gastrointestinal inflammation, radiation-induced gastrointestinal inflammation, immunotherapy-induced gastrointestinal inflammation, bacterial infection, viral infection, fungal infection, antibiotic use, aging, dysbiosis, obesity, type 2 diabetes, metabolic syndrome, asthma, atherosclerosis, non-alcoholic fatty liver disease, multiple sclerosis, or skin inflammation.

10. A tablet, chewable gum, capsule, granule, powder, sachet, patch, cream, or ointment comprising Cyberlindnera jadinii or Kluyveromyces lactis.

11. A kit comprising:
(a) a composition comprising Cyberlindnera jadinii or Kluyveromyces lactis; and
(b) written instructions for administration to a subject.

12. A method for treating and/or preventing gastrointestinal inflammation and/or a disease, disorder or condition associated with gastrointestinal inflammation, in a subject in need thereof, comprising administering a composition comprising an effective amount of Cyberlindnera jadinii or Kluyveromyces lactis to the subject.

13. (canceled)

14. The method of claim 12, wherein the disease, disorder, or condition is IBD, IBS, cancer, chemotherapy-induced gastrointestinal inflammation, radiation-induced gastrointestinal inflammation, immunotherapy-induced gastrointestinal inflammation, bacterial infection, viral infection, fungal infection, antibiotic use, aging, dysbiosis, obesity, type 2 diabetes, metabolic syndrome, asthma, atherosclerosis, non-alcoholic fatty liver disease, multiple sclerosis, or skin inflammation.

15. The method of claim 12, wherein the disease, disorder, or condition is IBD.

16. The method of claim 12, wherein the disease, disorder, or condition is ulcerative colitis or Crohn's disease.

17. The method of claim 12, wherein the disease, disorder, or condition is dysbiosis.

18. A kit comprising:
(a) the tablet, chewable gum, capsule, granule, powder, sachet, patch, cream, or ointment of claim 10; and
(b) written instructions for administration to a subject.
